(19) United States
(12) Patent Application Publication
(10) Pub. No.: US 2010/0285471 A1
(43) Pub. Date: Nov. 11, 2010

(54) Methods and Compositions for the Diagnosis and Treatment of Esphageal Adenocarcinomas

(75) Inventors: Carlo M. Croce, Columbus, OH (US); Curtis C. Harris, Garrett Park, MD (US); Ewy A. Mathe, Falls Church, VA (US)

Correspondence Address: MacMillan, Sobanski & Todd, LLC - NIH/OSU, One Maritime Plaza, 5th Floor, 720 Water Street, Toledo, OH, 43604, US

(73) Assignees: THE OHIO STATE UNIVERSITY RESEARCH FOUNDATION, Columbus, OH (US); The Government of the US of America as represented by any Secretary of the Dept. of Health, Rockville, MD (US)

(21) Appl. No.: 12/682,318

(22) PCT Filed: Oct. 10, 2008
(86) PCT No.: PCT/US08/79482

§ 371 Date: Jul. 7, 2010

Related U.S. Application Data
(60) Provisional application No. 60/979,300, filed on Oct. 11, 2007.

(51) Int. Cl. C12Q 1/68

(52) U.S. Cl. 435/6

(57) ABSTRACT

Methods and compositions for the diagnosis, prognosis and/or treatment of esophageal adenocarcinoma and Barrett's esophagus associated adenocarcinoma are disclosed, along with more markers where a difference is indicative of esophageal adenocarcinoma and squamous cell carcinoma, and/or Barrett's esophagus associated adenocarcinomas or a predisposition thereto. The invention also provides methods and compositions of identifying anti-cancer agents therefor.

### CROSS-REFERENCE TO RELATED APPLICATIONS

This application claims the benefit of U.S. Provisional Application No. 60/979,300 filed Oct. 11, 2007, the entire disclosure of which is expressly incorporated herein by reference.

### STATEMENT REGARDING FEDERALLY SPONSORED RESEARCH

This invention was made with government support under National Cancer Institute Grant No. ______. The government has certain rights in this invention.

### TECHNICAL FIELD AND INDUSTRIAL APPLICABILITY OF THE INVENTION

This invention relates generally to the field of molecular biology. More particularly, it concerns methods and compositions involving biomarkers for esophageal cancer and Barrett's esophagus. Certain aspects of the invention include application in diagnostics, therapeutics, and prognostics of Barrett's esophagus and esophageal cancer, including adenocarcinoma and squamous cell carcinoma.

### BACKGROUND OF THE INVENTION

There is no admission that the background art disclosed in this section legally constitutes prior art.

Esophageal cancer is the 8^{th} most common cancer and the 6^{th} most common cause of cancer deaths worldwide.¹ Often diagnosed at later stages, the survival rate for affected patients is very low, ranging from 10% in Europe² to 16% in the United States. The incidence of esophageal cancer varies greatly by geographical location, where it is most common in China, South East Africa, and Japan, and by gender, where males are affected more than females (7:1 ratio).⁴ In recent years though, the incidence of Barrett's esophagus associated adenocarcinoma, mainly caused by gastric reflux and obesity, has been rising, while the incidence of squamous cell carcinoma, mainly caused by cigarette and alcohol consumption, has been decreasing in the United States.⁴

Barrett's esophagus results from chronic gastro-esophageal reflux and is characterized by the replacement of normal esophageal squamous cell epithelium by metaplastic columnar epithelium. This chronic inflammatory condition is a well recognized precursor of esophageal adenocarcinomas.^{5,6} MiRNAs are small (20-24 nucleotides), well-conserved, non-coding RNA molecules that regulate the translation of MiRNAs.⁷⁻⁹ Since the discovery of the first miRNA, lin-4, in C. elegans in 1993¹⁰, the miRNA registry has housed sequences from 218 miRNAs in 2002 to 4584 in 2007, including miRNAs in primates, rodents, birds, fish, worms, flies, plants and viruses.^{11,12} In humans, over 300 miRNAs have been discovered. Mature miRNAs are generated from primary miRNA (pri-miRNA) molecules, containing a few hundred base pairs, which are further processed into pre-miRNAs by Drosha and Pasha in the nucleus.¹³⁻¹⁵ The pre-miRNAs are then exported in the cytoplasm and further processed into small, ˜22 nucleotides in length, RNA duplexes by Dicer.^{16,17}

The functional miRNA strand then binds within the RISC complex, which includes Dicer, TRBP, and Argonaute2 protein.^{3,18} In animals, this miRNA-RISC complex binds to its target MiRNA, via partial sequence complementarity, thereby blocking translation.¹⁹ Each miRNA is thought to play a role in the post-transcriptional regulation of hundreds of genes, and translation blocking of a given gene may require binding of more than one miRNA.¹⁹ This broad influence of miRNAs suggests their ubiquitous role and involvement in the large majority of genetic and disease pathways.

Recently, an increasing amount of studies have demonstrated the role of miRNAs in various human cancers²⁰ and have shown altered miRNA expression in most tumor types.^{21,22} In, addition, miRNAs are oftentimes located in fragile sites or cancer-associated genomic regions.^{23,24} The involvement of miRNAs in cancer was first reported in chronic lymphocytic leukemia, where mir-15 and mir-16 were down-regulated in ˜68% of the tumor cases.²⁵ Subsequent expression studies showed the involvement of mir-155²⁶ and the mir-17-92 locus²⁷ in B-cell lymphoma, reduced expression of mir-143 and mir-145 in colorectal cancer²⁸, over-expression of mir-21 in glioblastoma²⁹, and reduced expression of let-7 in lung cancer tissue and its association with survival.³⁰ Recently, we and others reported the involvement of let-7 and miR-155 in lung cancer diagnosis and prognosis (19-22)³¹ and high expression of miR-21 was associated with poor survival and therapeutic outcome in colon. [Schetter A, JAMA 2008, PMID: 18230780]. Other expression profiling studies allowed the identification of miRNA signatures in pancreatic cancer³², breast cancer³³, and papillary thyroid cancer.³⁴ Importantly, the successful use of antagomirs to silence miRNAs in mice.³⁵ and non-human primates [Elmen J, Nature 2008, PMID: 18368051] suggests the possible use of miRNAs in therapeutics

In the context of esophageal carcinoma, a recent study has shown an increased expression of RNASEN, a miRNA processing enzyme that acts at the level of the pri-miRNA to pre-miRNA conversion in the nucleus, in tumor samples of esophageal squamous cell carcinoma patients, suggesting the role of miRNA in esophageal tumor progression.³⁶ Recently, miRNA differential expression between squamous esophagus, Barrett's esophagus, cardia and cancer was reported, although their sample size was limited.³⁷

A better understanding of the biological mechanisms underlying esophageal adenocarcinoma is crucial for earlier diagnosis and more effective treatment options, in the hopes of increasing survival rates.

In spite of considerable research into therapies to treat these diseases, they remain difficult to diagnose and treat effectively, and the mortality observed in patients indicates that improvements are needed in the diagnosis, treatment and prevention of the disease.

### SUMMARY OF THE INVENTION

In a first broad aspect, there is provided herein a method for assessing a pathological condition in a subject which includes measuring an expression profile of one or more markers where a difference is indicative of esophageal cancers and inflammatory precursor conditions that can give rise to esophageal cancer or predisposition thereto.

In a broad aspect, there is provided herein a method of detecting one or more of esophageal adenocarcinoma, Barrett's esophagus and squamous cell carcinoma in a subject.

In another broad aspect, there is provided herein a method of early diagnosing a subject suspected of having esophageal adenocarcinoma, Barrett's esophagus or squamous cell carcinoma.

In still another broad aspect, there is provided herein a method of determining the likelihood of a subject to develop esophageal adenocarcinoma, Barrett's esophagus or squamous cell carcinoma.

These methods can include analyzing the sample for the altered expression of at least one biomarker associated with esophageal adenocarcinoma, Barrett's esophagus or squamous cell carcinoma, and correlating the altered expression of the at least one biomarker with the presence or absence of esophageal carcinoma, Barrett's esophagus or squamous cell carcinoma in the sample, wherein the at least one biomarker is selected from the group consisting of the mirs listed herein.

In certain embodiments, the biomarkers are detected in the sample using probes selected from the group consisting of one or more of the mir probes listed herein.

In certain embodiments, the correlation distinguishes between one or more of: 1) cancerous tissue (CT) and non-cancerous tissue (NCT) in adenocarcinoma (ADC) patients; 2) cancerous tissue (CT) and non-cancerous tissue (NCT) in adenocarcinoma (ADC) patients with Barrett's esophagus (BE); 3) Barrett's esophagus (BE) and non-Barrett's esophagus (NBE) in adenocarcinoma patients (ADC); 4) cancerous tissue (CT) and non-cancerous tissue (NCT) in squamous cell carcinoma (SCC); and 5) adenocarcinoma (ADC) and squamous cell carcinoma (SCC) in cancerous tissue (CT).

In certain embodiments, for correlation 1), the sample is analyzed for one or more of: the increased expression of at least one biomarker that is selected from the group consisting of mir-21, mir-223, mir-146a, mir-146b, and mir-181a; and/or the decreased expression of at least one biomarker that is selected from the group consisting of let-7c, mir-203 and mir-205.

In certain embodiments, for correlation 2), the sample is analyzed for one or more of: the increased expression of at least one biomarker that is selected from the group consisting of mir-21, mir-103, and mir-107; and/or the decreased expression of at least one biomarker that is selected from the group consisting of let-7c, mir-210, mir-203 and mir-205.

In certain embodiments, for correlation 3), the sample is analyzed for one or more of: the increased expression of at least one biomarker that is selected from the group consisting of mir-192, mir-215, mir-194, mir-135a, mir-92, mir-93, mir-7, mir-17, mir20b, mir-107, mir-103 and mir-191; and/or the decreased expression of at least one biomarker that is selected from the group consisting of mir-30b, mir-193a, let-7b, let-71, let-7d, let-7a, mir-369 and let-7c.

In certain embodiments, for correlation 4), the sample is analyzed for one or more of: the increased expression of at least one biomarker that is selected from the group consisting of mir-21, mir-223, mir-146b, mir-224, mir-155, mir-7-2, mir-181b, mir-146a, mir-181, mir-7, mir-16, mir-122a, mir-125a, and mir-16; and/or the decreased expression of at least one biomarker that is selected from the group consisting of mir-202, mir-29c, mir-30b, mir-30c, mir-126, mir-99a, mir-220, mir-320, mir-499, mir-30c, mir-125b, mir-1, mir-145, mir-143, mir-378, mir-200b, mir-133a, mir-375 and mir-203.

In certain embodiments, for correlation 5), the sample is analyzed for one or more of: the increased expression of at least one biomarker that is selected from the group consisting of mir-215, mor-192 and mir-194; and/or the decreased expression of at least one biomarker that is selected from the group consisting of mir-142, mir-224 and mir-155.

The sample can be blood or tissue, and in certain embodiments, the tissue is esophageal tissue. The tissue can be selected from the group consisting of tumor tissue, nontumor tissue, and tissue adjacent to a tumor.

In yet another broad aspect, there is provided herein a method of treating a subject with esophageal carcinoma, Barrett's esophagus or squamous cell carcinoma, comprising administering a therapeutically effective amount of a composition comprising a nucleic acid complementary to at least one biomarker selected from the group consisting of the mirs listed herein.

In another broad aspect, there is provided herein a pharmaceutical composition comprising a nucleic acid complementary to at least one biomarker selected from the group consisting of the mirs listed herein.

In another broad aspect, there is provided herein a method of comparing adenocarcinoma tissue samples that have undergone chemoradiation therapy and carcinoma tissue samples that have not undergone chemoradiation therapy, comprising comparing differential expression of at least one of the mirs listed herein.

In another broad aspect, there is provided herein a method of comparing nodal involvement in squamous cell carcinoma tissue samples, comprising comparing differential expression of at least one of the mirs listed herein.

In another broad aspect, there is provided herein a method of comparing staging in squamous cell carcinoma tissue samples, comprising comparing differential expression of at least one of the mirs listed herein.

In another broad aspect, there is provided herein a method of diagnosing whether a subject has, or is at risk for developing, esophageal carcinoma, Barrett's esophagus or squamous cell carcinoma, comprising measuring the level of at least one mir in a test sample from the subject, wherein an alteration in the level of the mir in the test sample, relative to the level of a corresponding mir in a control sample, is indicative of the subject either having, or being at risk for developing, esophageal adenocarcinoma, Barrett's esophagus or esophageal squamous cell carcinoma; wherein the mir is selected from one or more of the mir listed herein.

In another broad aspect, there is provided herein a method for suppressing esophageal adenocarcinoma, Barrett's esophagus or esophageal squamous cell carcinoma in a subject in need thereof, comprising administering at least one gene selected from the group consisting of the mirs listed herein.

In another broad aspect, there is provided herein a method of diagnosing an esophageal adenocarcinoma, Barrett's esophagus or esophageal squamous cell carcinoma related disease associated with one or more prognostic markers in a subject, comprising measuring the level of at least one mir in a sample from the subject, wherein an alteration in the level of the at least one mir in the test sample, relative to the level of a corresponding mir in a control sample, is indicative of the subject having an esophageal adenocarcinoma, Barrett's esophagus or esophageal squamous cell carcinoma related disease associated with the one or more prognostic markers; wherein the mir is selected from the group consisting of the mirs listed herein.

In another broad aspect, there is provided herein a method of diagnosing whether a subject has, or is at risk for developing, esophageal adenocarcinoma, Barrett's esophagus or esophageal squamous cell carcinoma, comprising: 1) reverse transcribing RNA from a test sample obtained from the subject to provide a set of target oligodeoxynucleotides; 2) hybridizing the target oligodeoxynucleotides to a microarray comprising miRNA-specific probe oligonucleotides to provide a hybridization profile for the test sample; and 3) comparing the test sample hybridization profile to a hybridization profile generated from a control sample, wherein an alteration in the signal of at least one mir is indicative of the subject either having, or being at risk for developing, an esophageal adenocarcinoma, Barrett's esophagus or esophageal squamous cell carcinoma related disease; wherein the mir is selected from the group consisting of the mirs listed herein. In certain embodiments, the signal of at least one mir, relative to the signal generated from the control sample, is down-regulated. In certain other embodiments, the signal of at least one mir, relative to the signal generated from the control sample is up-regulated.

In another broad aspect, there is provided herein a method of treating an esophageal carcinoma, Barrett's esophagus or squamous cell carcinoma related disease in a subject suffering therefrom in which at least one mir is down-regulated or up-regulated in the cancer cells of the subject relative to control cells, comprising: 1) when the at least one mir is down-regulated in the cancer cells, administering to the subject an effective amount of at least one isolated mir, such that proliferation of cancer cells in the subject is inhibited; or 2) when the at least one mir is up-regulated in the cancer cells, administering to the subject an effective amount of at least one compound for inhibiting expression of the at least one mir, such that proliferation of cancer cells in the subject is inhibited; wherein the mir is selected from the group consisting of the mirs listed herein.

In another broad aspect, there is provided herein a method of treating esophageal carcinoma related disease in a subject, comprising: 1) determining the amount of at least one mir in esophageal cells, relative to control cells, wherein the mir is selected from the group consisting of the mirs listed herein; and 2) altering the amount of mir expressed in the esophageal cells by: (i) administering to the subject an effective amount of at least one isolated mir, if the amount of the mir expressed in the esophageal cells is less than the amount of the mir expressed in control cells; or (ii) administering to the subject an effective amount of at least one compound for inhibiting expression of the at least one mir, if the amount of the mir expressed in the esophageal cells is greater than the amount of the mir expressed in control cells, such that proliferation of esophageal adenocarcinoma, Barrett's esophagus or esophageal squamous cell carcinoma cells in the subject is inhibited.

In another broad aspect, there is provided herein a method of identifying an anti-esophageal related disease agent, comprising providing a test agent to an esophageal cell and measuring the level of at least one mir associated with decreased expression levels in the esophageal cell, wherein an increase in the level of the mir in the esophageal cell, relative to a suitable control cell, is indicative of the test agent being an anti-cancer agent; wherein the mir is selected from the group consisting of the mirs listed herein.

In another broad aspect, there is provided herein a method for assessing a pathological condition, or the risk of developing a pathological condition, in a subject comprising: measuring an expression profile of one or more markers in a sample from the subject, wherein a difference in the expression profile in the sample from the subject and an expression profile of a normal sample is indicative of esophageal adenocarcinoma, Barrett's esophagus or esophageal squamous cell carcinoma or a predisposition thereto, wherein the marker at least comprises one or more mirs listed herein.

In another broad aspect, there is provided herein a composition comprising one or more of the mirs is selected from the group consisting of the mirs listed herein.

In another broad aspect, there is provided herein a reagent for testing for an esophageal adenocarcinoma, Barrett's esophagus or esophageal squamous cell carcinoma, wherein the reagent comprises a polynucleotide comprising the nucleotide sequence of at least one mir listed herein, or a nucleotide sequence complementary to the nucleotide sequence of the marker.

In another broad aspect, there is provided herein a reagent for testing for an esophageal adenocarcinoma, Barrett's esophagus or esophageal squamous cell carcinoma, related disease, wherein the reagent comprises an antibody that recognizes a protein encoded by at least one mir listed herein.

In another broad aspect, there is provided herein a method of assessing the effectiveness of a therapy to prevent, diagnose and/or treat an esophageal adenocarcinoma, Barrett's esophagus or esophageal squamous cell carcinoma, comprising: 1) subjecting an animal to a therapy whose effectiveness is being assessed, and 2) determining the level of effectiveness of the treatment being tested in treating or preventing an esophageal adenocarcinoma, Barrett's esophagus or esophageal squamous cell carcinoma, by evaluating at least one mir listed herein. In certain embodiments, the candidate therapeutic agent comprises one or more of: pharmaceutical compositions, nutraceutical compositions, and homeopathic compositions. In certain embodiments, the therapy being assessed is for use in a human subject.

In another broad aspect, there is provided herein an article of manufacture comprising: at least one capture reagent that binds to a marker for an esophageal adenocarcinoma, Barrett's esophagus or esophageal squamous cell carcinoma related disease selected from at least one of the mir listed herein.

In another broad aspect, there is provided herein a kit for screening for a candidate compound for a therapeutic agent to treat an esophageal adenocarcinoma, Barrett's esophagus or esophageal squamous cell carcinoma related disease, wherein the kit comprises: one or more reagents of at least one mir listed herein, and a cell expressing at least one mir. In certain embodiments, the presence of the mir is detected using a reagent comprising an antibody or an antibody fragment which specifically binds with at least one mir.

In another broad aspect, there is provided herein a screening test for an esophageal adenocarcinoma, Barrett's esophagus or esophageal squamous cell carcinoma related disease comprising: contacting one or more of the mirs listed herein with a substrate for such mir and with a test agent, and determining whether the test agent modulates the activity of the mir. In certain embodiments, all method steps are performed in vitro.

In another broad aspect, there is provided herein use of an agent that interferes with an esophageal adenocarcinoma, Barrett's esophagus or esophageal squamous cell carcinoma related disease response signaling pathway, for the manufacture of a medicament for treating, preventing, reversing or limiting the severity of a an esophageal adenocarcinoma, Barrett's esophagus or esophageal squamous cell carcinoma related disease complication in an individual, wherein the agent comprises at least one mir listed herein.

In another broad aspect, there is provided herein a method of treating, preventing, reversing or limiting the severity of an esophageal adenocarcinoma, Barrett's esophagus or esophageal squamous cell carcinoma related disease complication in an individual in need thereof, comprising administering to the individual an agent that interferes with at least an esophageal adenocarcinoma, Barrett's esophagus or esophageal squamous cell carcinoma related disease response cascade, wherein the agent comprises at least one mir listed herein.

In another broad aspect, there is provided herein use of an agent that interferes with at least an esophageal adenocarcinoma, Barrett's esophagus or esophageal squamous cell carcinoma related disease response cascade, for the manufacture of a medicament for treating, preventing, reversing or limiting the severity of a cancer-related disease complication in an individual, wherein the agent comprises at least one mir listed herein.

In another broad aspect, there is provided herein novel methods and compositions for the diagnosis, prognosis and treatment of esophageal cancers and inflammatory precursor conditions. The invention also provides methods of identifying anti-esophageal cancer agents and anti-inflammatory precursor agents.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file contains one or more drawings executed in color and/or one or more photographs. Copies of this patent or patent application publication with color drawing(s) and/or photograph(s) will be provided by the Patent Office upon request and payment of the necessary fee.

FIGS. 1A-1B: Kaplan-Meier Analysis depicting associations with qRT-PCR miRNA expression and survival. MiRNA expression values were dichotomized into low and high groups, using the within cohort median expression value as a cutoff.

FIG. 1A: Associations observed in ADC patients without Barrett's esophagus. Reduced expression of mir-203 in NCT (N=11) is associated with worse prognosis. Survival profiles were compared using the log rank test with P<0.05 indicating statistical significance.

FIG. 1B: Associations observed in SCC patients. In NCT, elevated expression of mir-21 (N=35), mir-155 (N=35), mir-146b (N=35), and mir-181b (N=35) is associated with worse prognosis while reduced expression of mir-375 (N=35) in CT is associated with worse prognosis.

FIG. 1C: Ratios of differentially expressed miRNAs, showing fold changes <0.75 or >1.25. Differential microarray expression between cancerous (CT) and non-cancerous tissue (NCT) in adenocarcinoma patients (1), CT and NCT in ADC patients with Barrett's esophagus (BE) (2), CT tissue of BE and non-BE (NBE) in ADC patients (3), CT and NCT in SCC patients (4), CT tissue of ADC and SCC patients (5). The color scale corresponds to the microarray expression fold changes.

FIG. 2: qRT-PCR validation of differentially expressed miRNAs when comparing cancerous and non-cancerous tissue. Relative log expression differences between cancerous (CT) and non-cancerous (NCT) in the ADC (a) and in SCC (b) patients. All expression values are normalized to RNAU66. In ADC patients, mir-375 differential expression in both sets and mir-194 differential expression in training set samples were borderline statistically significant (0.005<P<0.05) while all others were statistically significant (P<0.005). In SCC patients, mir-181b, mir-155, and mir-146b differential expression in validation set samples and mir-203 differential expression in training set samples were borderline statistically significant while all other alterations were statistically significant.

FIG. 3: qRT-PCR validation of differentially expressed mirs when comparing Barrett's Esophagus (BE) and Non-Barrett's Esophagus (NBE) in the cancerous tissue of adenocarcinoma cases. Relative log expression differences between BE and NBE in cancerous tissue. All expression values are normalized to RNAU66 and all differential expression represented are borderline statistically significant (0.005<P<0.05).

FIG. 4: qRT-PCR validation of mirs with altered expression in cancerous tissue between ADC and SCC patients. Relative log expression differences between ADC and SCC patients in cancerous tissue. All expression values are normalized to RNU66 and altered expression depicted here are statistically significant (P<0.005), except for mir-375 in the training set (0.05<P<0.005).

FIG. 5: Table 1: Patient clinical, pathological and demographic characteristics.

FIG. 6: Table 2: Differential microarray expression of mir probes in the Training set.

FIG. 7: Table 3: Univariate and multivariate Cox modeling to assess associations between mir qRT-PCR expression levels and survival.

FIG. 8: Supplemental Table 1 showing differentially expressed probed (P<0.05 and DRF<10%) that represent mature mirs, according to the microarray expression, when comparing CT and NCT tissue in adenocarcinoma samples.

FIG. 9: Supplemental Table 2: Differentially expressed probes (P<0.05 and FDR<10%) that represent mature mirs, according to microarray expression, when comparing CT and NCT tissue in adenocarcinoma/Barrett's esophagus samples.

FIG. 10: Supplemental Table 3: Differentially expressed probes (P<0.05 and FDR<10%) that represent mature mirs, according to microarray expression, when comparing Barrett's esophagus (BE) and non-Barrett's esophagus (NBE) adenocarcinoma tissue.

FIG. 11: Supplemental Table 4: Differentially expressed probes (P<0.05 and FDR<10%) that represent mature mirs, according to microarray expression, when comparing adenocarcinoma tissue samples that have undergone chemoradiation therapy (CRT) and those that have not (nCRT).

FIG. 12: Supplemental Table 5: Differentially expressed probes (P<0.05 and FDR<10%) that represent mature mirs, according to microarray expression, when comparing CT and NCT tissue in squamous cell carcinoma samples.

FIG. 13: Supplemental Table 6: Differentially expressed probes (P<0.05 and FDR<10%) that represent mature mirs, according to microarray expression, when comparing nodal involvement (N=0 vs. N=1) in squamous cell carcinoma tissue.

FIG. 14: Supplemental Table 7: Differentially expressed probes (P<0.05 and FDR<10%) that represent mature mirs, according to microarray expression, when comparing staging (TNM stage 0-I vs. II-IV) in squamous cell carcinoma tissue. Relative log expression differences between ADC and SCC patients in cancerous tissue. All expression values are normalized to RNU66 and altered expression depicted here are statistically significant (P<0.005), except for mir-375 in the training set (0.05<P<0.005).

FIG. 15: Supplemental Table 8: Differentially expressed probes (P<0.05 and FDR<10%) that represent mature mirs, according to microarray expression, when comparing ADC and SCC samples in cancerous tissue.

FIG. 16: Supplemental Table 9: Classification of samples into their diagnosis, BE status, and histological categories, using miRNA microarray expression profiles.

FIG. 17: Supplemental Table 10: List of persistent miRNA probes used in the final PAM classification models using miRNA microarray expression.

FIG. 18: Supplemental Table 11: Detailed univariate and multivariate Cox models.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference.

MiRNA expression levels, measured using miRNA microarrays³⁸, of tumor (CT) and adjacent non-cancerous (NCT) tissue pairs were used to evaluate expression differences between CT and NCT tissue, and Barrett's esophagus (BE) and non-Barrett's esophagus (NBE) tissue. Expression differences of select mature miRNAs were validated using qRT-PCR in an independent cohort comprising CT/NCT pairs. Furthermore, we evaluated the utility of miRNAs as predictive biomarkers of clinico-pathological outcome, including diagnosis, prognosis, and Barrett's status.

In addition to studying esophageal adenocarcinoma, miRNA expression in squamous cell carcinoma has been evaluated. We have identified and confirmed the differential expression between cancerous and non-cancerous tissue miRNAs in adenocarcinoma and squamous cell carcinoma patients, and successfully used miRNA profiles to predict diagnosis, Barrett's esophagus status, and histological type. Significantly, we identified miRNAs associated with survival, independent of other known prognostic clinical parameters. Thus, we have demonstrated a link between miRNAs, esophageal carcinoma, and inflammation, and provided preliminary evidence for their potential clinical utility as early diagnostic and prognostic biomarkers. These miRNAs may furthermore be utilized as potential targets for novel personalized drug therapies.

MicroRNA expression levels associated with prognosis can be further used for in situ hybridization of tissue microarrays. This technique also allows for high-throughput analysis, and allows researchers to assess whether it can improve the prognostic utility of microRNA biomarkers. With the ambiguity and uncertainty in the staging of esophageal adenocarcinoma, miRNA prognostic predictors can greatly aid in the choice of therapy. In addition, functional assays in human cell lines, whereby specific miRNAs can be knocked in or knocked out, can be used to evaluate changes in tumor and Barrett's esophagus phenotype.

Esophageal adenocarcinoma is often detected at later stages and is most often associated with poor prognosis. Potential miRNA biomarkers that may predispose individuals to Barrett's esophagus and/or esophageal adenocarcinoma could provide a means for earlier detection and help in better identifying treatment options. Furthermore, antagomirs have been successfully used to silence miRNAs in vivo, thereby making it feasible to regulate the expression of cancer-associated genes. This application thus opens avenues for the possible use of miRNAs in identifying novel drug targets and therapies.

The inventors further demonstrate herein the involvement of miRNAs in the pathogenesis of human esophageal cancers and Barrett's esophagus in a large cohort, and explored their association with survival.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference.

### EXAMPLES

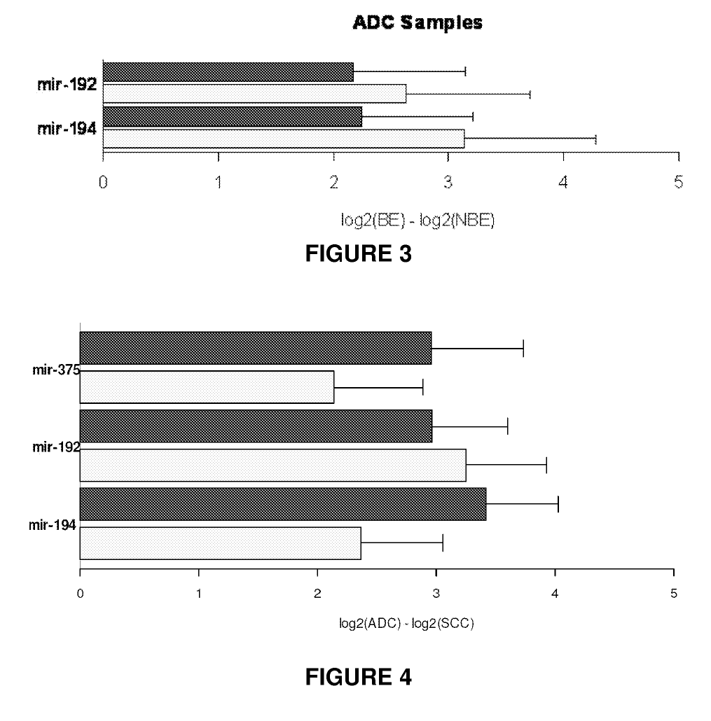

Using cancerous (CT) and non-cancerous (NCT) tissue resected from patients split into training and validation sets, we first generated miRNA microarray³⁵ profiles and subsequently confirmed expression differences of relevant miRNAs using qRT-PCR in all samples. Clinical characteristics of all patients are summarized in FIG. 5—Table 1.

While no differences between clinical variables in both cohorts were observed for ADC patients, differences in gender and stage were observed between cohorts of SCC patients. MicroRNA microarray expression values were first evaluated in training set samples and subsequently confirmed using qRT-PCR in all samples.

MicroRNA Differential Expression in ADC Cases.

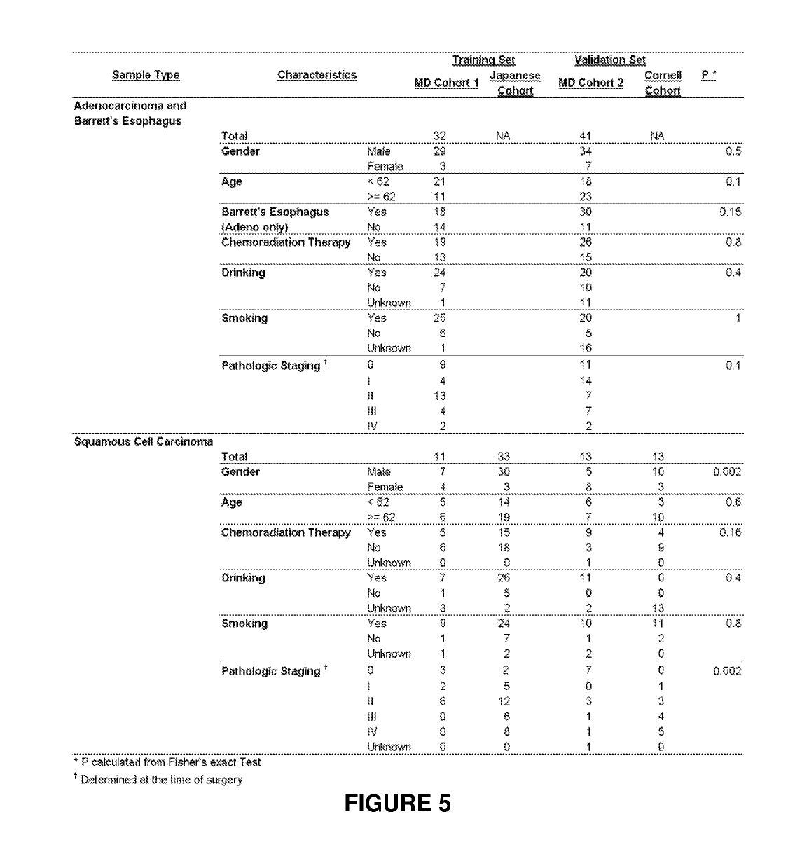

Alterations in miRNA microarray expression levels specific to ADC patients were evaluated in 32 CT and adjacent NCT pairs. The top panel of FIG. 6—Table 2 lists differentially expressed miRNAs (P<0.05, FDR<10%) whose probes contain the mature miRNA sequence. Increased expression was observed for miR-21, miR-223, miR-146a, miR-146b, and miR-181a, and decreased expression was detected for miR-203 and miR-205. When Barrett's esophagus associated ADC patients were assessed, miR-21, miR-103, miR-107, and let-7c exhibit increased expression while miR-210, miR-203, and miR-205 show reduced expression. MiRNAs with altered expression were not identified in patients with sporadic ADC. Expression between Barrett's esophagus associated and sporadic ADC CT is increased in miR-192, miR-215, miR-194, miR-135a and decreased in a number of miRNAs belonging to the let-7 family.

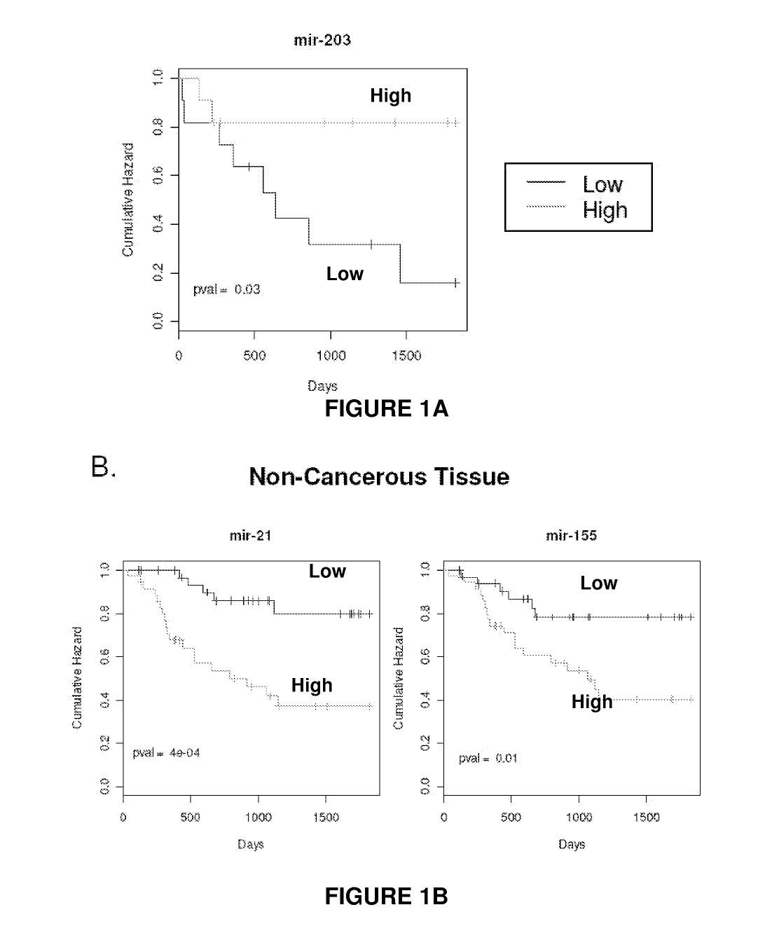

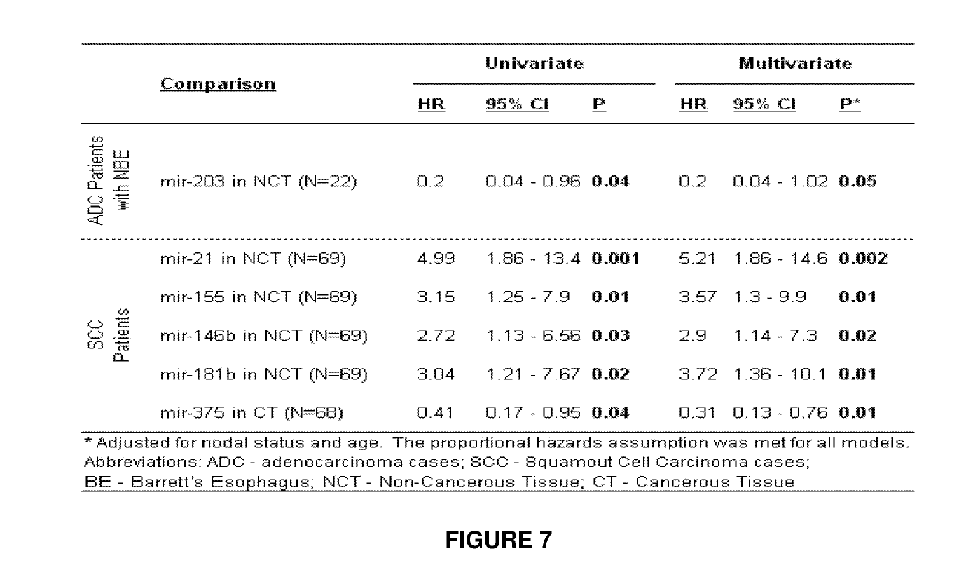

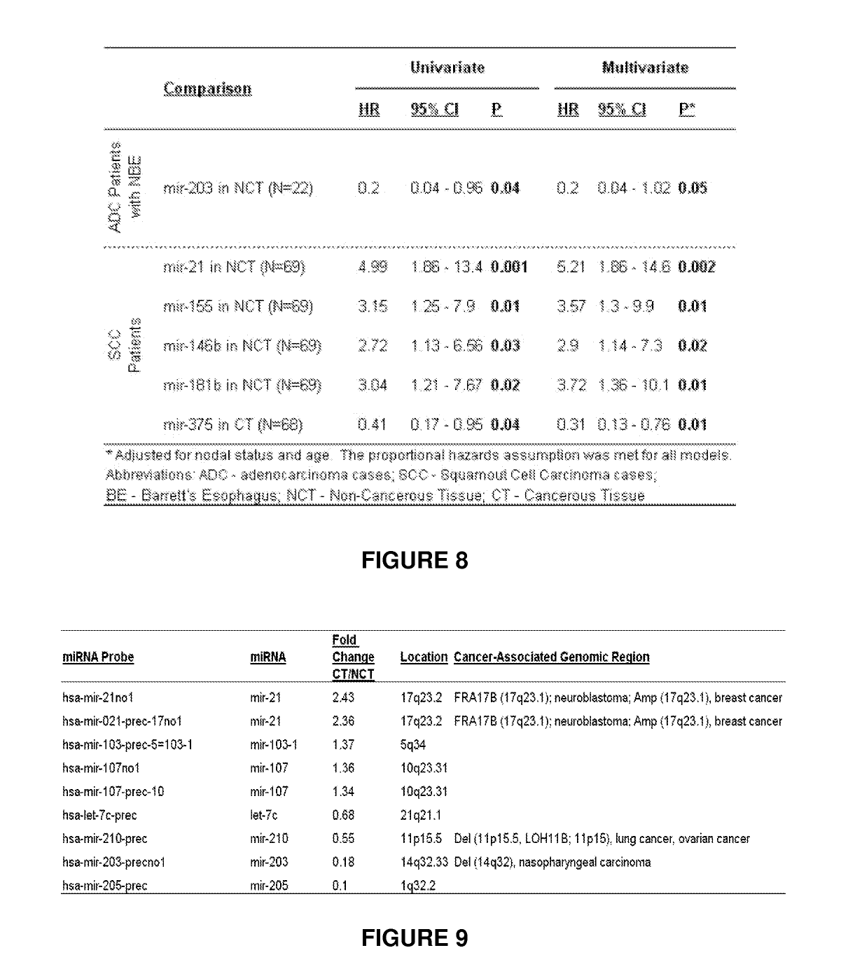

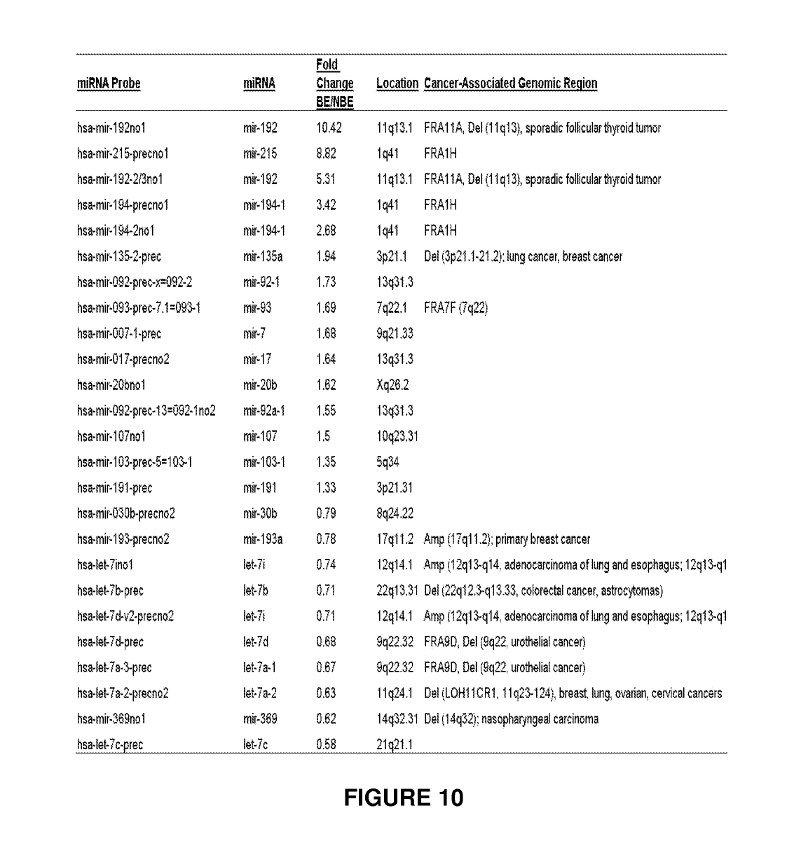

Many of the differentially expressed probes are located in fragile sites and Cancer Associated Genomic Regions (FIG. 8—Supplemental Table 1, FIG. 9—Supplemental Table 2, FIG. 10—Supplemental Table 3). A visual representation of miRNA fold changes for these comparisons is depicted in FIG. 1C.

Expression levels of let-7a and let-7c measured in a subset of training set samples using qRT-PCR did not show concordance with microarray results. Nonetheless, these miRNAs may warrant further studies since they are located in fragile sites or Cancer Associated Genomic Regions. Furthermore, let-7 has been found to repress tumor formation in the lung of mice and an association between reduced expression of let-7 and survival has been demonstrated in human lung cancer tissue. Differential expression was also observed between cancerous tissue of ADC patients that had and had not undergone neo-adjuvant chemoradiation therapy. Because therapy was administered prior to tissue collection, it was not possible to directly link these affected miRNAs with therapy. No differential expression was observed when evaluating age, nodal involvement, stage, smoking status, and alcohol consumption.

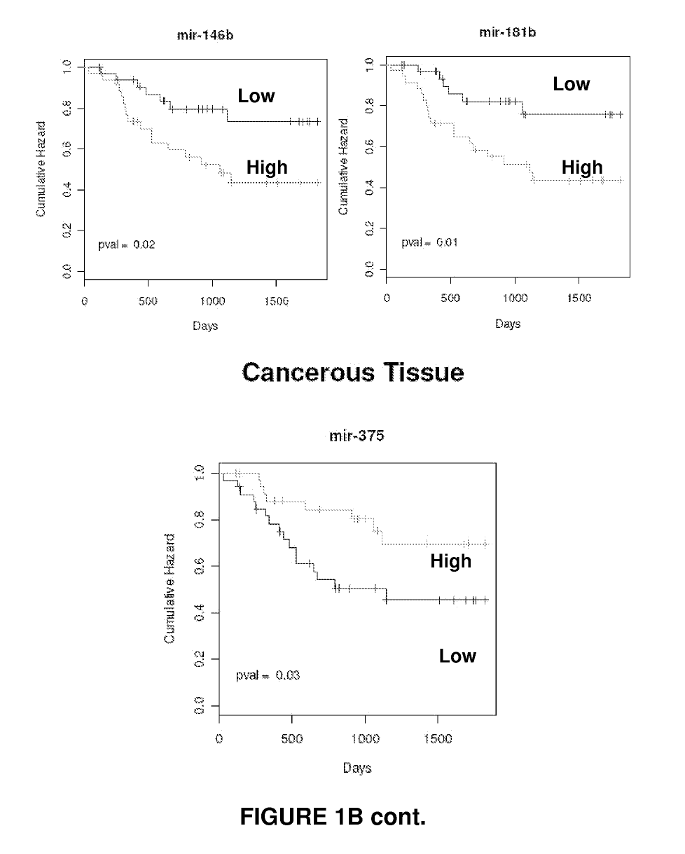

Expression measurements of select miRNAs (P<0.05, FDR<10%, and largest fold changes) were validated using qRT-PCR. Elevated expression of miR-21, miR-223, and reduced expression of miR-203 and miR-375 in ADC cancerous compared to adjacent non-cancerous tissue was confirmed in training and validation set samples (FIG. 2a).

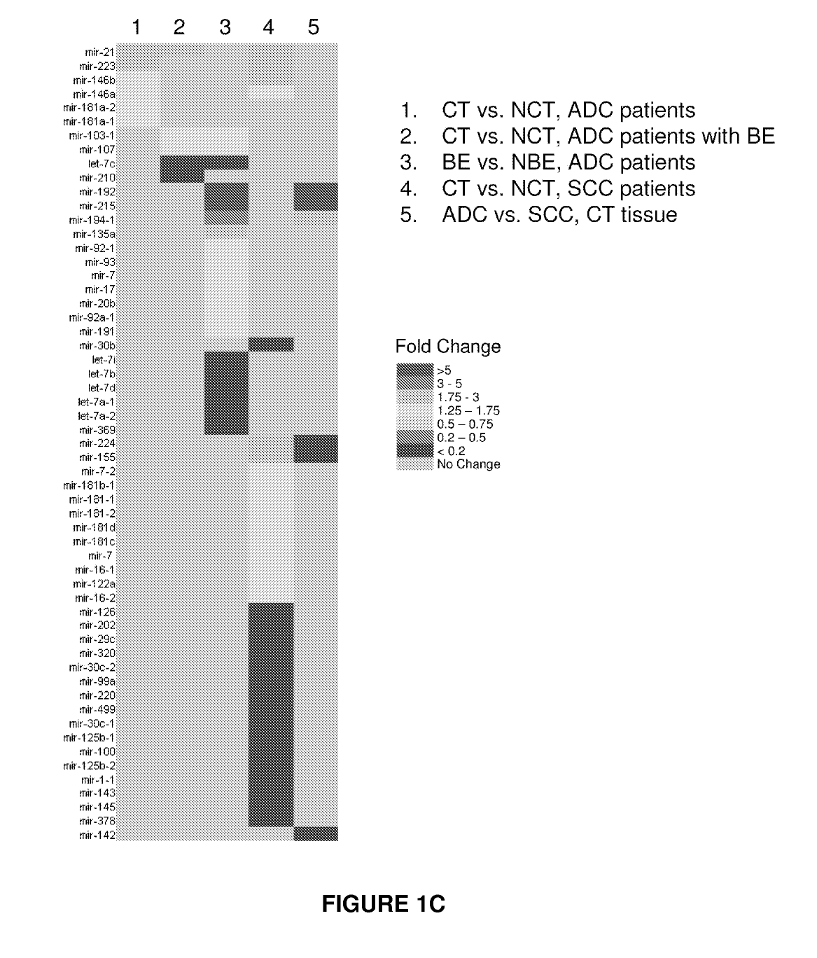

In addition, altered expression of miR-194 and miR-192 in cancerous tissue between Barrett's esophagus associated and sporadic ADC patients was validated (FIG. 3). Increased expression of these two miRNAs was also increased in cancerous compared to non-cancerous tissue, although these changes were not statistically significant in the microarray analysis. Furthermore, ADC patients with Barrett's esophagus showed increased expression of miR-21, miR-192, miR-194, and reduced expression of miR-203 in cancerous compared to non-cancerous tissue. Altered expression of these miRNAs was also present in patients without Barrett's esophagus although statistical significance was not attained, perhaps due to small sample size (N=14).

Association Between miRNA Expression and Survival.

For greater ease of interpretation, miRNA expression values derived from qRT-PCR were dichotomized based on a within cohort median cutoff (see METHODS herein).

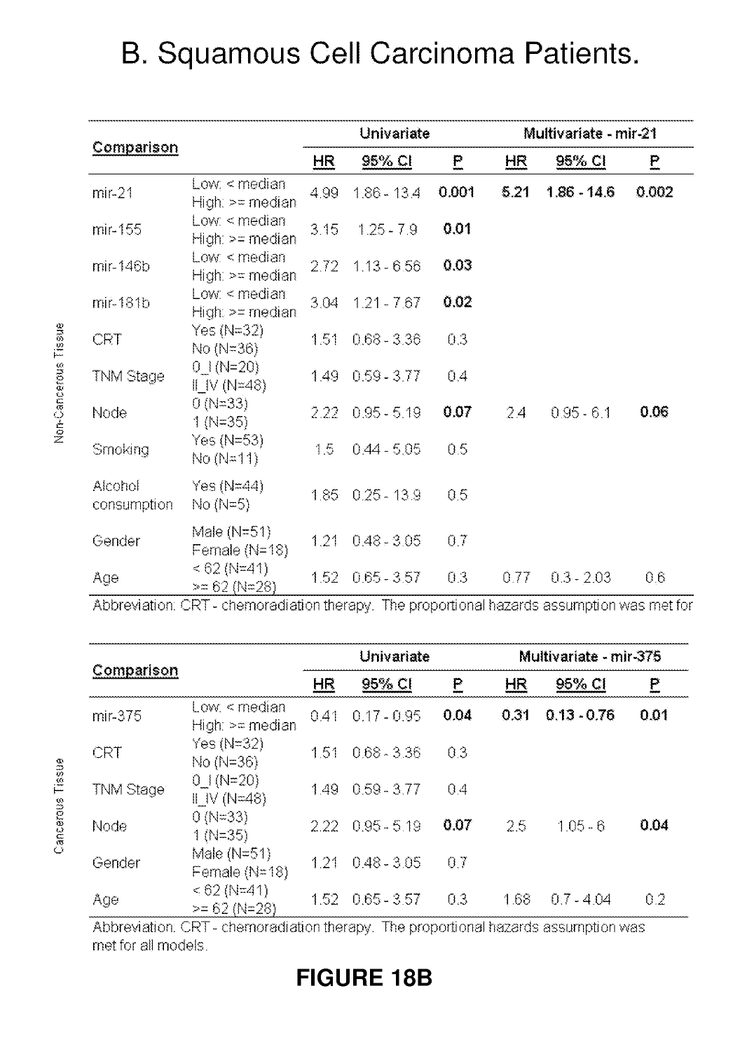

Associations between miRNA expression and survival were not observed in ADC patients (N=73). When evaluating ADC patients without Barrett's esophagus, low expression of miR-203 in non-cancerous tissue (N=22) was borderline associated (HR=0.2; 95% confidence interval [CI]=0.04−0.96) with poor prognosis, independent of nodal status and age (HR=0.2; 95% CI=0.04−1.02) (FIG. 5—Table 3, FIG. 18 Table 11b).

MiRNA expression of ADC patients also diagnosed with Barrett's esophagus showed no statistically significant association with survival.

MicroRNA Differential Expression in SCC Cases.

Altered miRNA expression specific to SCC was next sought in 44 patients. When comparing cancerous and adjacent non-cancerous tissue, increased expression was observed in miR-21, miR-223, miR-146b, miR-224, miR-155, miR-181b, miR-146a, and reduced expression was detected in miR-203, miR-375, and miR-133a (P<0.05 and FDR<10%) (see FIG. 6—Table 2).

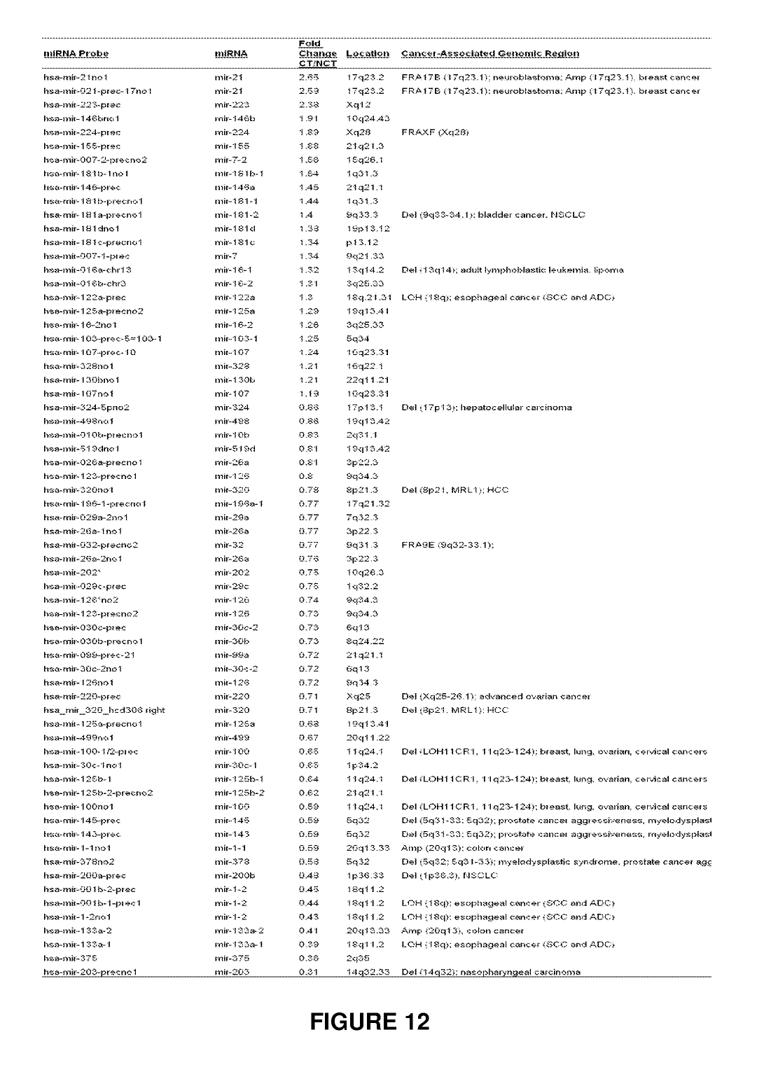

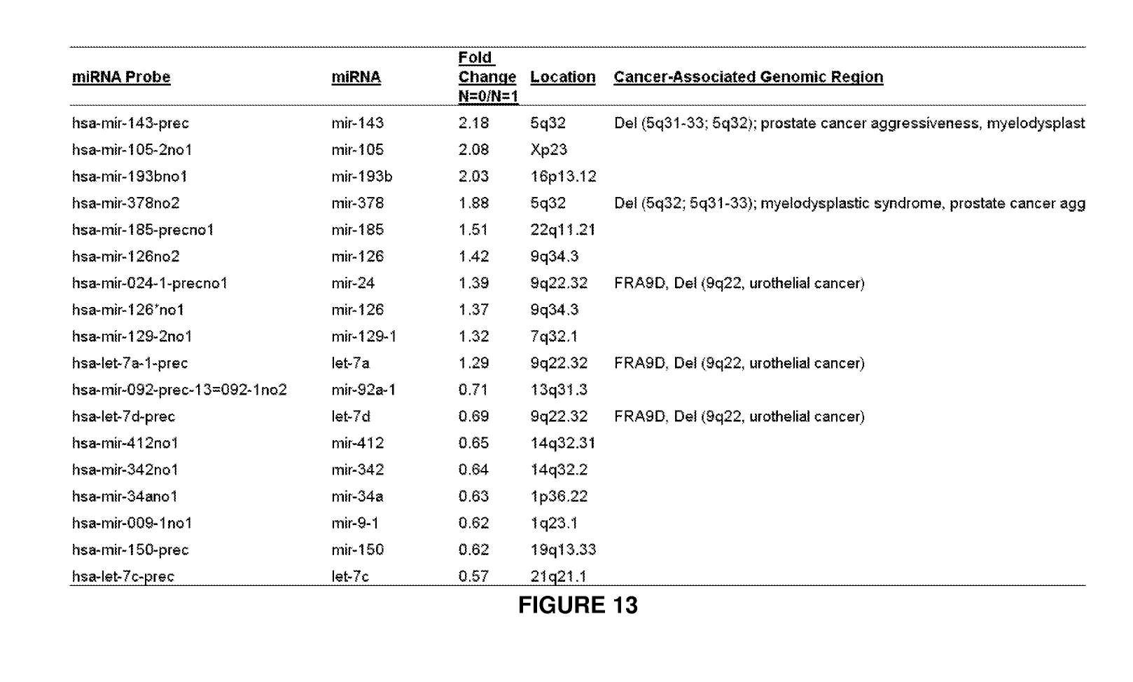

Thirty-five percent of the probes are located in Cancer Associated Genomic Regions (FIG. 12—Supplemental Table 5). Altered expression was not observed when comparing age, administration of neo-adjuvant chemoradiation therapy, smoking and alcohol consumption status. However, altered expression in the non-cancerous tissue of patients with nodal involvement and in patients with low pathologic TNM stage was observed (FIG. 13—Supplemental Table 6). A visual summary of fold changes for differentially expressed miRNAs is shown in FIG. 1C.

Expression measurements were confirmed using qRT-PCR in all available cases, including 26 additional validation set samples. Elevated expression levels of miR-21, miR-181b, miR-155, and miR-146b and reduced levels of miR-203, miR-375 were confirmed when comparing cancerous and adjacent non-cancerous tissue (FIG. 2b). Interestingly, elevated levels of miR-21, miR-203, and levels of miR-375 in cancerous tissue were also observed in ADC samples, suggesting that expression of these miRNAs may be altered in esophageal carcinoma, regardless of histological type.

Association Between miRNA Expression and Survival.

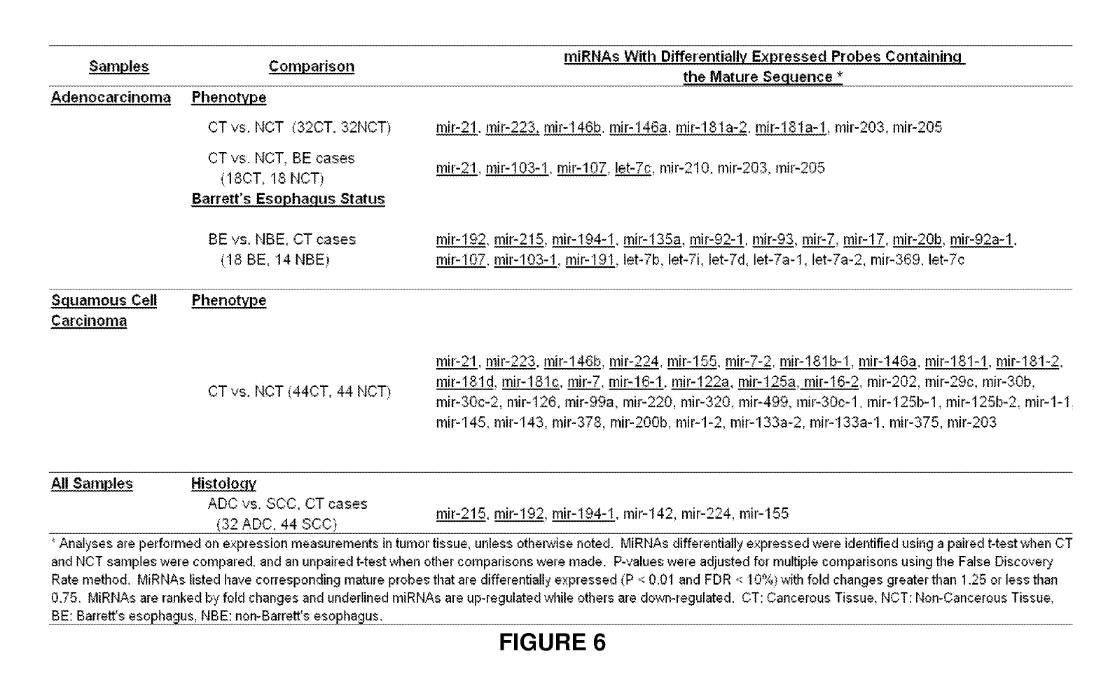

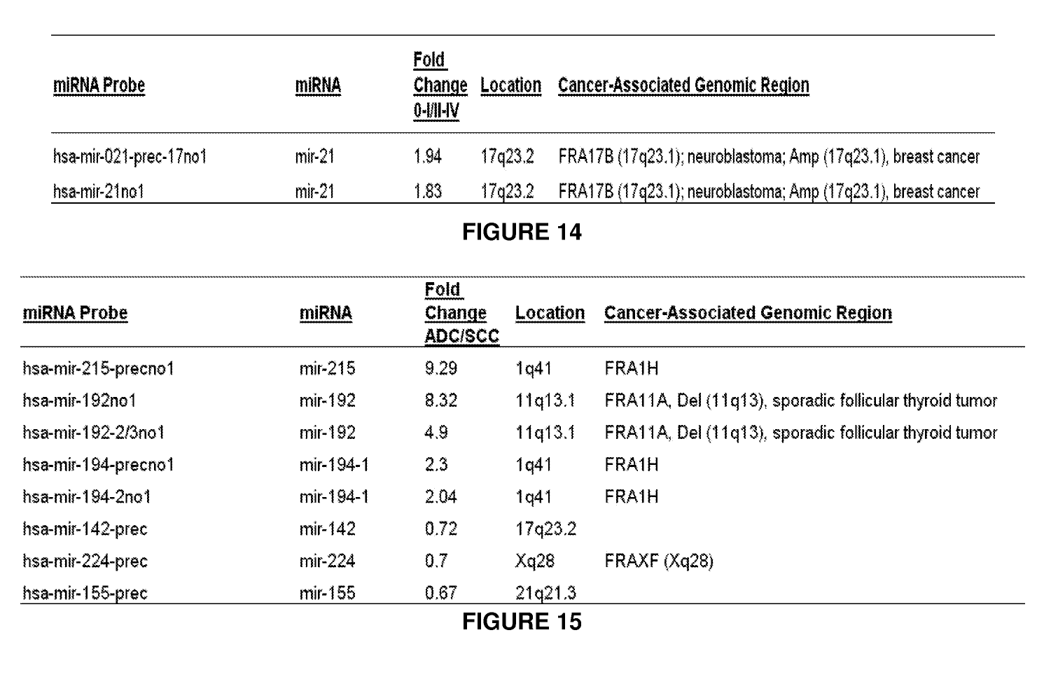

Similar to the analysis of ADC patients, qRT-PCR expression values were dichotomized based on a median cutoff within each cohort. Kaplan-Meier analysis revealed a statistically significant association between high expression of miR-21 in non-cancerous tissue (HR=4.99; 95% CI=1.86−13.4) and worse prognosis (FIG. 1, FIG. 7—Table 3, FIG. 14—Supplemental Table 7).

Elevated levels of miR-155 (HR=3.15; 95% CI=1.25−7.9), miR-146b (HR=2.72; 95% CI=1.13−6.56), and miR-181b (HR=3.04; 95% CI=1.21−7.67) in non-cancerous tissue showed a borderline significant association with worse prognosis. Furthermore, reduced miR-375 expression (HR=0.41; 95% CI=0.17−0.95) in tumor tissue was borderline associated with poor prognosis. Multivariate Cox modeling revealed that associations between miRNA expression and survival are independent of nodal involvement and age.

MicroRNA Differential Expression Between ADC and SCC Patients.

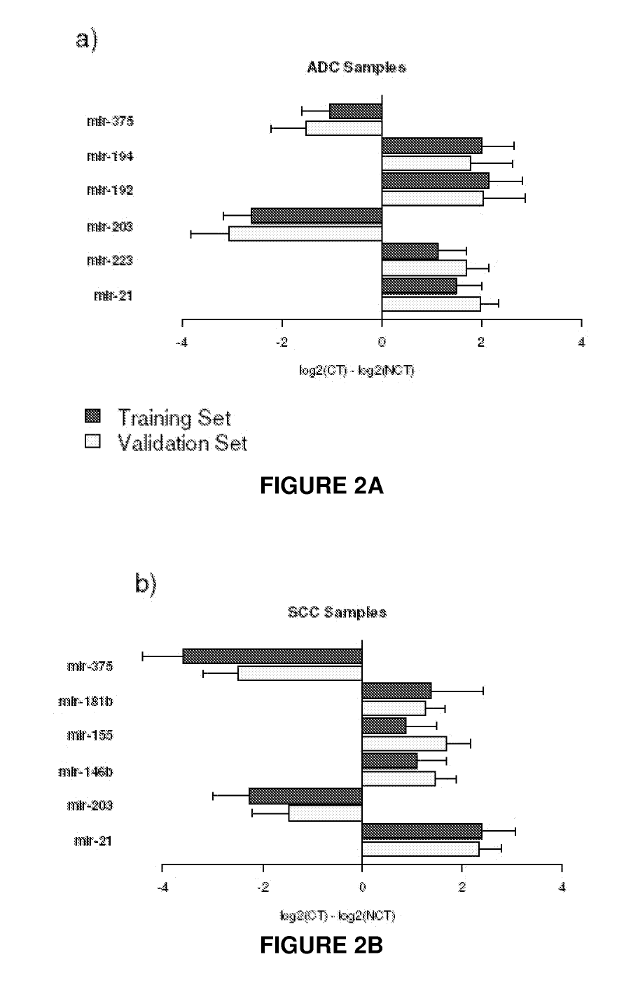

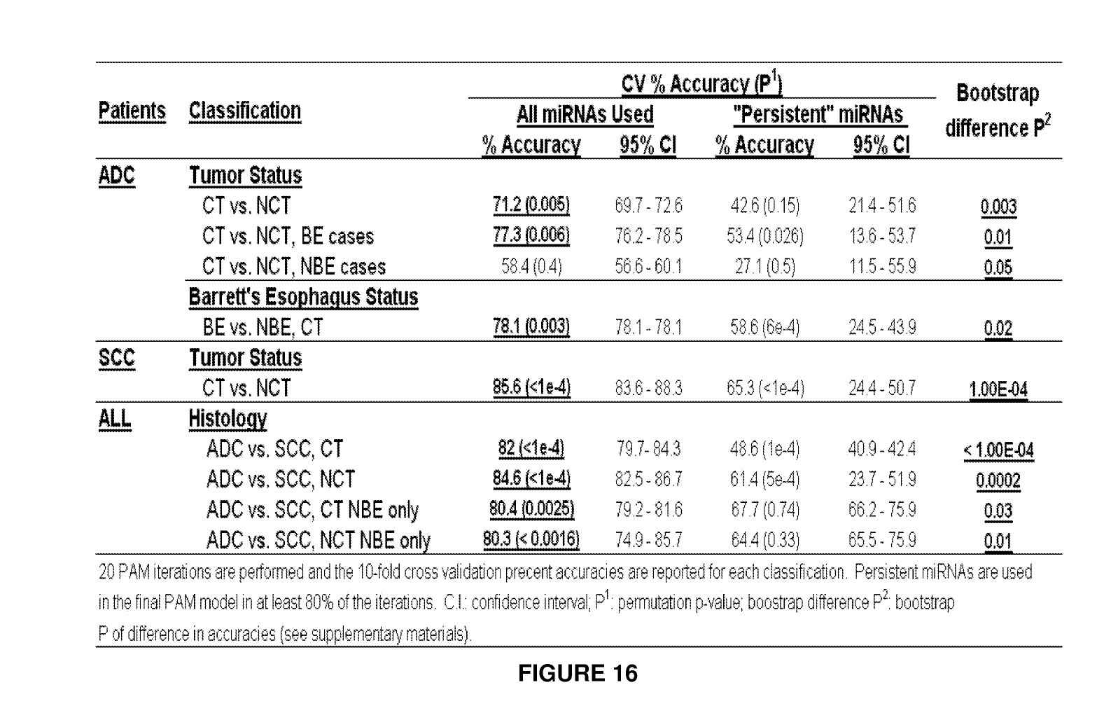

When comparing histological types in cancerous tissue, increased expression of miR-215, miR-192, miR-194, and reduced expression of miR-155, miR-224, and miR-142 are observed in ADC compared to SCC patients (FIG. 4. Table 2, FIG. 15—Supplemental Table 8 and FIG. 1C).

Differential expression was not detected in non-cancerous tissue, suggesting that adjacent non-cancerous tissue have similar miRNA profiles, regardless of histological type. When only considering patients without Barrett's esophagus, increased expression of miR-192 and decreased expression of miR-155 in SCC cases was observed (P<0.05), although FDRs were elevated (>50%). Elevated expression levels in cancerous tissue of miR-194 and miR-192 in ADC compared to SCC patients were confirmed using qRT-PCR (FIG. 4).

Increased expression of miR-375 in the cancerous tissue of ADC compared to SCC patients was also observed in our validation. Altered expression in these miRNAs suggests that the underlying biological mechanisms in cancerous cells may differ between the two different histological types, and that treatment therapies specific to each histological type may be more efficient.

Sample Classification Using miRNA Microarray Expression.

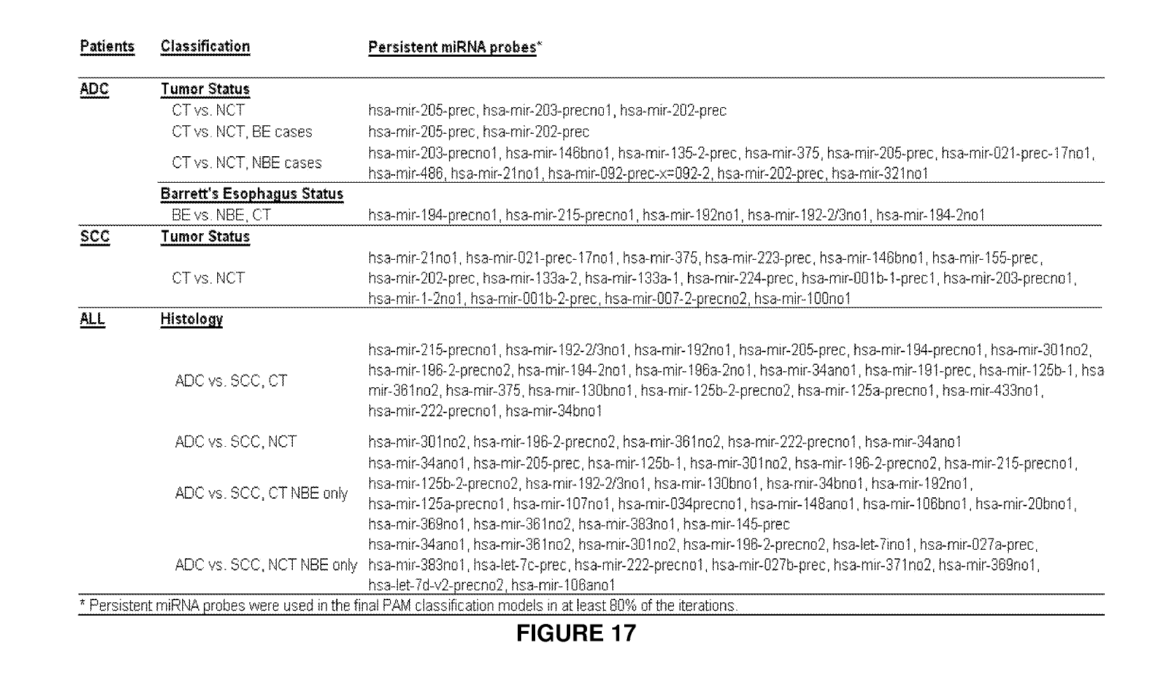

Samples were classified by tumor status and types by inputting miRNA microarray expression values in Prediction Analysis of Microarrays (FIG. 16—Supplemental Table 9). When classifying ADC samples, 71% accuracy (P=0.005) was obtained when discerning cancerous from adjacent non-cancerous tissue. Using Barrett's esophagus associated ADC patients increased the accuracy to 77% (P=0.006) while using patients with sporadic ADC yielded near random class assignment (58% accuracy), analogous to the differential expression analysis described above. Furthermore, 78% accuracy (P=0.003) was obtained when classifying cancerous tissue expression of Barrett's esophagus associated or sporadic ADC patients. Expectedly, random classification accuracies were obtained when expression in non-cancerous tissue were input. Classification of SCC samples into cancerous and non-cancerous tissue yielded 86% accuracy (P<1e−4), which was substantially higher than that obtained when classifying ADC samples in their diagnostic categories (71.2%).

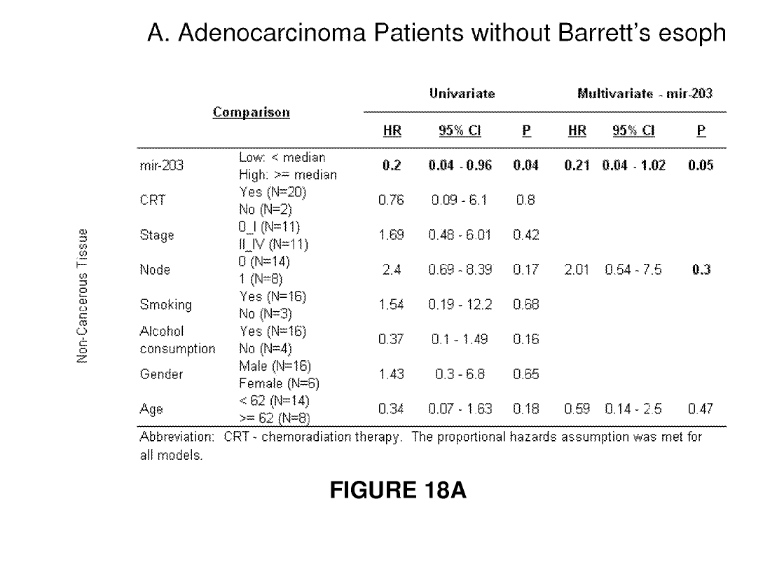

This finding suggests that miRNA profiles of ADC cases are more heterogeneous than those of SCC cases, which may partly be due to differences in Barrett's esophagus status. Finally, classification of samples by histology yielded 82% and 85% accuracies using cancerous and non-cancerous tissue expression profiles, respectively. Importantly, there is a large overlap between “persistent” miRNA probes, which contribute most to the classifications, and those that showed differential expression (FIG. 17—Supplemental Table 10). In all cases, differences in accuracies between models that use all probes and models built after removing “persistent” probes are statistically significant (P<0.05).

Differential Expression Between Clinical Characteristics.

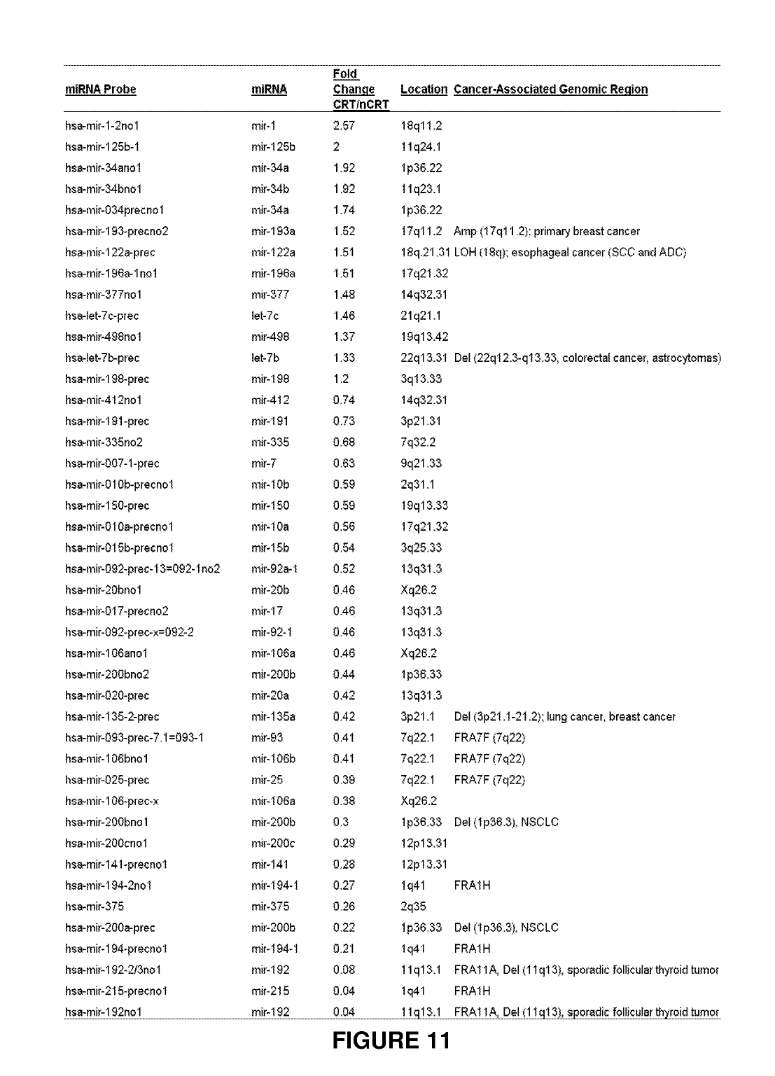

Using microarray measurements, altered expression of 43 mature miRNA probes was observed in the cancerous tissue of adenocarcinoma (ADC) patients that underwent neo-adjuvant chemoradiation therapy compared to those that did not (FIG. 11—Supplemental Table 4). However, differential expression was not observed when comparing neo-adjuvant chemoradiation therapy status in non-cancerous tissue. These observations suggest that miRNA expression may be affected by neo-adjuvant chemoradiation therapy in cancer cells but not in the adjacent non-cancerous tissue. Furthermore, miRNAs altered by therapy in cancerous tissue may be indicators of tumors that are resistant to therapy. However, these hypotheses can only be verified by comparing cancerous and adjacent non-cancerous tissue both prior to and after the administration of chemotherapy. In all these cases, chemoradiation therapy was administered prior to surgery, and therefore prior to sample collection.

In SCC patients, when comparing expression levels of non-cancerous tissue of patients with or without nodal involvement, 19 probes showed differential expression (FIG. 13—Supplemental Table 6). However, no probes were altered when expression levels in cancerous tissue was evaluated. This observation suggests a possible association between miRNA regulation and lymph node involvement, albeit the lack of this observation in ADC cases. Nonetheless, differential expression was also observed in non-cancerous tissue of lower stage cases with the tumor restricted to the lining of the esophagus (TNM stage 0-I) versus higher stage cases (TNM stage II-IV). More specifically, probes including the mature sequence of mir-21 show elevated levels in lower stage cases (FIG. 14—Supplemental Table 7). Similar to nodal status, changes in expression were not observed between stages in cancerous tissue.

In all cases, when comparing patients that had undergone chemoradiation therapy and those that had not, differential expression was observed in cancerous tissue (FIG. 18—Supplemental Table 11) but not in non-cancerous tissue. Again, because therapy was administered prior to surgical resection, it is difficult to directly assess whether the expression differences are solely due to therapy. As was observed in SCC patients, expression of mir-21 probes were altered between cases with lower staging (TNM 0 to I) and those with higher staging (TNM II-IV) in non-cancerous tissue.

Discussion

The Examples herein describe a study that assesses the potential diagnostic and prognostic utility of miRNAs in esophageal cancer. MiRNA expression was evaluated in 143 cancerous and adjacent non-cancerous tissue pairs and we identified miRNAs important for classification of samples into diagnostic and Barrett's esophagus categories.

Elevated miR-21 and reduced miR-203 and miR-375 levels were observed in both SCC and ADC samples, independently, indicating that these miRNAs may be involved in esophageal carcinogenesis, independent of histological type.

In cancerous tissue, increased expression of miR-194, miR-192, and miR-223 are observed in ADC patients while increased expression of miR-181b, miR-155, and miR-146b are detected in SCC patients.

Altered expression of these miRNAs is specific to histological type, suggesting a potential utility of histology-specific therapies to improve prognosis.

Expression levels of miRNAs mentioned above were validated in all samples using qRT-PCR. Over-expression of miR-21 and miR-155 is of great interest since they are ubiquitously induced in solid tumors, including lung, breast, stomach, prostate, colon, pancreas and in chronic lymphocytic leukemia. MiR-155 expression is also elevated in Burkitt's and B cell lymphomas, and is induced in response to macrophage driven inflammation in mice, thereby linking the roles of miR-155 in inflammation and cancer. MiR-21 targets tumor and metastasis suppressor genes, including phosphatase and tensin homolog PTEN, tumor suppressor gene tropomyosin 1 TPM1, programmed cell death 4 PDCD4, and Sprouty2, thereby demonstrating its involvement in tumor growth, invasion, and metastasis.

Also, miR-155 is a prognostic predictor in lung cancer and that elevated miR-21 cancerous/non-cancerous ratio expression levels are associated with poor prognosis and therapeutic outcome in colon cancer. Furthermore, miR-181b is differentially expressed in chronic lymphocytic leukemia and negatively regulates the expression of the oncogene Tcl1, and miR-146b is induced by pro-inflammatory cytokines and plays a role in Toll-like receptor and cytokine signaling. These and other results demonstrate a regulatory interplay between miRNAs and inflammatory cytokines.

The inventors herein now demonstrate here that altered levels of miR-21 in non-cancerous tissue of SCC patients are associated with survival, suggesting that miR-21 may have an indirect effect in SCC tumors. We have previously established that the combination of cytokine expression in non-cancerous and cancerous tissue of lung ADC patients are predictors of survival, suggesting a possible interaction between the tumor and its surrounding lung environment. Furthermore, there is growing evidence for the role of miRNAs in regulating innate and acquired immune response. Specifically, mir-21 expression has been associated with immune-related diseases, including B-cell lymphoma and chronic lymphocytic leukemia. Furthermore, a recent study demonstrated the Stat3-dependent effect of interleukin-6 on miR-21 induction, which contributed to the oncogenic potential of Stat3. Consequently, our finding that increased levels of miR-21 are associated with worse prognosis in non-cancerous tissue is possibly a reflection of an immune response that is associated with tumorigenesis.

In concordance with our observations, a recent study based on a cohort of 7 patients reported that miR-21 is over-expressed in ADC, miR-143 is under-expressed in ADC, and miR-194 is over-expressed in Barrett's esophagus (30). The study also reported over-expression of miR-203, miR-205, miR-143, and miR-215 in Barrett's esophagus, which we did not observe in our analysis.

Also concordant with our results, another study reported the analysis of 20 cases and 9 normal epithelial tissue and revealed an over-expression of miR-21 and under-expression of miR-203 and miR-205 in cancerous tissue in both histological subtypes (70).

In a previous study evaluating miRNA expression in SCC patients, high expression of miR-103 and miR-107 correlated with poor survival in 30 patients, a finding confirmed in an independent set of 22 SCC patients (71). These results were not in concordance with our analysis, perhaps due to their use of a different microarray platform and more limited sample size.

The administration of neo-adjuvant chemoradiation therapy (prior to surgery) in 54% of patients used in this example and complete pathologic response in 22% of patients limits our ability to negate the role of therapy on associations between miRNA expression and diagnosis/prognosis. Of note, patients with complete pathologic response are not necessarily cured, perhaps due to remaining systemic processes or the inability to detect small metastatic disease (72). These patients' survival rate is worse than that of the general population and it is still a debate whether such patients have longer survival than patients without complete pathologic response (73). This observation further demonstrates the importance of identifying molecular biomarkers, such as miRNAs, that would help refine staging and predict treatment response. Furthermore, while chronic alcohol consumption and smoking may adversely affect survival of esophageal cancer patients (74) (75), we were unable to adequately assess the influence of those covariates in our multivariate Cox analysis due to missing values (16% and 23% missing values for smoking and alcohol consumption, respectively).

These Examples demonstrate the role of miRNAs in esophageal cancer and identify miRNAs whose expression is altered in and between SCC and ADC cancerous tissue, and in cancerous tissue between Barrett's associated and sporadic ADC cancerous tissue.

These Examples also show the association between elevated miR-21 levels in non-cancerous tissue with worse prognosis, thereby suggesting a possible association between miR-21, immune response, and SCC. Prognostic association of miRNA expression in non-cancerous tissue is of particular interest because altered levels of these miRNAs may be evident prior to advanced disease stage and the occurrence of symptoms. MiRNA expression levels of less invasive tissue biopsies may be used to assess who may or may not benefit from surgical resection of the esophagus, which is a very invasive procedure. The ability to block miRNA transcription may open avenues for the possible use of miRNAs in identifying novel drug targets and therapies for esophageal carcinoma.

Materials and Methods

Clinical Samples.

A total of 143 patients with available cancerous and adjacent non-cancerous tissue from surgical resection were divided into a training and a validation set. The training set includes 44 SCC cases and 32 ADC cases, of which 18 were also diagnosed with Barrett's esophagus, while the validation set comprises 26 SCC cases and 41 ADC cases, including 30 patients also diagnosed with Barrett's esophagus. Patients were recruited from 3 different cohorts: (1) University of Maryland Medical System in Baltimore, Md., (2) Nippon Medical School in Tokyo, Japan, (3) New York Presbyterian-Weill Cornell Medical Center in NY, US. Samples collected from the Maryland Cohort were divided into two groups: MD Cohort 1 was included in the training set while MD Cohort 2 was included in the validation set (FIG. 5—Table 1).

Disease stage and survival were obtained from medical records, pathology reports, State of Maryland records, and the National Death Index. These studies were approved by the Institutional Review Boards of the participating institutions. Clinico-pathological data relevant to this study were provided from their respective sources and include gender, age, histology, presence/absence of Barrett's esophagus, neo-adjuvant chemoradiation therapy administration, alcohol consumption, smoking status, and pathologic staging (FIG. 5—Table 1).

RNA Isolation and Quantification of miRNA.

Total RNA used for quantification of miRNA levels was extracted from esophageal tissue in our laboratory using TRIZOL (Invitrogen, cat. no. 15596-026), according to the manufacturer's procedures. MiRNA expression levels were measured using miRNA microarray chips version 3 (Ohio State University) containing 329 human miRNAs and 249 mouse miRNA probes in duplicate (1). Five μg of total RNA were converted to biotin-labeled first strand cDNA, hybridized onto the chips, and processed by direct detection of the biotin-containing transcripts by streptavidin-Alexa 647 conjugate. Slides were subsequently scanned with the Axon 4000B Scanner (Molecular Device, Inc.) and spot intensities were quantified with Genepix (version Pro 6.0.1.00). Microarray data is currently being submitted to the Gene Expression Omnibus, in compliance with MIAME guidelines.

Validation of miRNA altered levels was performed by qRT-PCR using Taqman miRNA reverse transcription assays (Applied Biosystems, cat. no. 4366596) and appropriate primers, following the manufacturer's instructions. In brief, 10 ng of total RNA was used as a template for a 15 μl reverse transcription reaction using probes specially designed for specific mature miRNAs. For each miRNA, reactions were performed in triplicate using the 7500 RT-PCR system (Applied Biosystems) and RNU66 (Applied Biosystems, cat. no. 4373382) was used as a control.

Statistical Analysis.

Differential Expression.

Pre-processing and normalization of miRNA microarray expression values were performed in R (version 2.6.0), a free software environment for statistical computing and graphics (2), and differential expression analysis was carried out in BRB ArrayTools (version 3.5.0) developed by Dr. Richard Simon and Amy Peng Lam (http://linus.nci.nih.gov/BRB-ArrayTools.html). Using R, mean intensity spot values for each sample were extracted for spots that were not flagged by the image quantification software GenePix (version Pro 6.0.1.00). In addition, spots were removed if their background intensities were higher than their respective foreground intensities, and if quadruplicate intensity spot values differed by more than 1 (on a log 2 scale). The remaining spots were then normalized using a loess normalization modified for single channel array data, where the true spot intensity is estimated by the average of that spot across all arrays. A loess curve is fit through (z˜‘means’) for each array, where z is the intensity of each spot in a given array, and means is the estimated true spot intensity. The normalized spot intensity is then obtained by subtracting the predicted value (obtained from the fitted loess curve) from the actual spot intensity.

After averaging duplicate spot intensity values, the normalized data was input into BRB ArrayTools (version 3.6.0) and subsequent analyses were restricted to human miRNA probes with intensity values present in at least 25% of the samples. Altered expression of miRNA probes was determined using the Class Comparison Tool, which performs t-tests, and expression changes with a P<0.05 and corresponding False Discovery Rate<10% were considered to be statistically significant. A paired t-test was performed when comparing cancerous and adjacent non-cancerous tissue expression, while a t-test with a random block design by date was applied for all other comparisons. The random block design by date controls for possible date bias to ensure that the differential expression was not confounded by the date at which the microarrays were hybridized and scanned.

qRT-PCR was utilized to validate microarray expression measurements of 13 miRNAs in 10% of randomly selected training set samples. Expression counts were normalized to RNU66 counts. We first asserted that these measurements were concordant (statistically significant and same-direction fold changes) with those from the microarrays in the training set samples. Next, we measured expression in the independent validation set samples to further verify expression changes. Concordance between both measurements (statistically significant and same direction fold changes) was established for 9 miRNAs, whose expression was subsequently measured in all remaining samples using qRT-PCR. Expression counts were normalized to RNU66 counts and two-sided paired or unpaired t-tests (for comparing cancerous and adjacent non-cancerous tissue, and all other comparisons, respectively) were performed.

Survival Analysis.

For ease of interpretation, miRNA expression values were dichotomized into high and low using median expression value within each cohort (i.e. MD cohorts, Japan cohort, and Cornell cohort) as a cutoff. Kaplan-Meier curves were constructed and survival differences were assessed using the Mantel-Haenszel or log rank test. To test the proportional hazards assumption, the R function cox.zph( ) was utilized, which correlates scaled Schoenfeld residuals with a suitable transformation of time. Univariate and multivariate Cox analysis was performed to assess associations between clinical variables and prognosis, and to adjust for relevant clinical variables.

Multivariate Cox models included clinical covariates that were either associated with survival in the univariate analysis or known as important clinical variables from previous publications. Specifically, nodal involvement, which has previously been shown to be associated with survival (3), and age were included in the final multivariate models.

To ensure a sufficient number of events per group, validation and testing cohorts were combined. While the hazard ratios show the same trend in both cohorts for a given miRNA when analyzed separately, P values exceeded 0.05 (data not shown), most likely due to an insufficient number of events per strata. Importantly, for all statistically significant associations between miRNA expression and survival, no differences in survival were observed between patients that showed complete pathological response and those that did not. Statistical significance of expression validation and survival analysis was achieved when P<0.005 (corresponding to P<0.05 after applying the stringent Bonferroni correction for 9 multiple comparisons) and borderline statistical significance was achieved when 0.005<P<0.05.

Classification.

Sample classification by tumor status, histology, and Barrett's esophagus status were performed using the R package “pamr” (version 1.34.0), Prediction Analysis of Microarrays (PAM) (4). Missing intensity values were imputed using the package routine “pamr.knnimpute”, which uses a nearest neighbor averaging algorithm. Twenty iterations of PAM were run, and for each iteration, the 10-fold cross validation (CV) accuracies were calculated. In addition, the list of miRNA probes used in the final model for each iteration were recorded, and “persistent” miRNA probes are defined as those that appear in the final model in at least 80% of the iterations. To further evaluate the importance of the persistent probes for classification, they were removed from the total probe list and the 20 iterations were repeated. Similarly, the 10-fold CV percent accuracies were recorded for the models built using this reduced set of probes, and these accuracies were compared to those obtained from models built using all the probes.

Two tests were performed to evaluate the robustness of the models. First, bootstrap techniques were used to estimate a distribution of CV percent accuracies from re-sampling with replacement of the original data (10,000 iterations). To evaluate whether the percent accuracies obtained were not random, the probability of obtaining a percent accuracy less than or equal to 50% was estimated from the bootstrap distribution. Second, to assess the difference in accuracies observed between the models using all probes and the models using all but the persistent probes, the probability of obtaining accuracy differences less than or equal to zero was calculated from the bootstrap estimated distribution. The reported confidence intervals were calculated from the bootstrap estimated distribution.

Vastly different number of samples in each class can cause artificially high accuracies. To correct this artifact, a prior probability correction for each class was applied to the PAM models, which essentially inflates the discriminant score between a new sample and different classes and balances the true and false positive rates. To determine the optimal set (for each class) of priors, we ran PAM using different sets of priors (i.e. [0,1], [0.01, 0.99], . . . , [1,0]) and constructed Receiving Operating Curves, which plot the False Positive Rates vs. True Positive Rates. The optimal prior set was objectively determined by identifying the point that lied on the convex hull, while minimizing the False Positive Rates and maximizing the True Positive Rates.

### Examples of Uses

In one aspect, the present invention provides methods for predicting survival of a subject with cancer. The prediction method is based upon the differential expression of a plurality of mirs as biomarkers in cancer cells. It is to be understood that the term “biomarkers” can be interchanged with the terms “mir”, mirs”, “miRs”, miRNAs, and “gene products”.

It was discovered that some biomarkers tend to be over-expressed, whereas other biomarkers tend to be under-expressed. The unique pattern of expression of these biomarkers in a sample of cells from a subject with cancer may be used to predict relative survival time, and ultimately the prognosis, for that subject.

A Method for Predicting Survival of a Subject with Cancer

One aspect of the invention provides a method for predicting cancer survival. The method comprises determining the differential expression of at least one, or in certain embodiments, a plurality of, biomarkers in a sample of cells from a subject with cancer. The biomarker expression signature of the cancer may be used to derive a risk score that is predictive of survival from that cancer. The score may indicate low risk, such that the subject may survive a long time (i.e., longer than 5 years), or the score may indicate high risk, such that the subject may not survive a long time (i.e., less than two years).

Survival-Related Biomarkers

Some of the biomarkers are over-expressed in long-term survivors and some of the biomarkers are over-expressed in short-term survivors. A biomarker may play a role in cancer metastasis by affecting cell adhesion, cell motility, or inflammation and immune responses. A biomarker may also be involved in apoptosis. A biomarker may play a role in transport mechanism. A biomarker may also be associated with survival in other types of cancer.

Measuring Expression of a Plurality of Biomarkers

One includes measuring the differential expression of a plurality of survival-related biomarkers in a sample of cells from a subject with cancer. The differential pattern of expression in each cancer—or gene expression signature—may then be used to generate a risk score that is predictive of cancer survival. The level of expression of a biomarker may be increased or decreased in a subject relative to other subjects with cancer. The expression of a biomarker may be higher in long-term survivors than in short-term survivors. Alternatively, the expression of a biomarker may be higher in short-term survivors than in long-term survivors.

The differential expression of a plurality of biomarkers may be measured by a variety of techniques that are well known in the art. Quantifying the levels of the messenger RNA (MiRNA) of a biomarker may be used to measure the expression of the biomarker. Alternatively, quantifying the levels of the protein product of a biomarker may be to measure the expression of the biomarker. Additional information regarding the methods discussed below may be found in Ausubel et al., (2003) Current Protocols in Molecular Biology, John Wiley & Sons, New York, N.Y., or Sambrook et al. (1989). Molecular Cloning: A Laboratory Manual, Cold Spring Harbor Press, Cold Spring Harbor, N.Y. One skilled in the art will know which parameters may be manipulated to optimize detection of the MiRNA or protein of interest.

A nucleic acid microarray may be used to quantify the differential expression of a plurality of biomarkers. Microarray analysis may be performed using commercially available equipment, following manufacturer's protocols, such as by using the Affymetrix GeneChip® technology (Santa Clara, Calif.) or the Microarray System from Incyte (Fremont, Calif.). Typically, single-stranded nucleic acids (e.g., cDNAs or oligonucleotides) are plated, or arrayed, on a microchip substrate. The arrayed sequences are then hybridized with specific nucleic acid probes from the cells of interest. Fluorescently labeled cDNA probes may be generated through incorporation of fluorescently labeled deoxynucleotides by reverse transcription of RNA extracted from the cells of interest. Alternatively, the RNA may be amplified by in vitro transcription and labeled with a marker, such as biotin. The labeled probes are then hybridized to the immobilized nucleic acids on the microchip under highly stringent conditions. After stringent washing to remove the non-specifically bound probes, the chip is scanned by confocal laser microscopy or by another detection method, such as a CCD camera. The raw fluorescence intensity data in the hybridization files are generally preprocessed with the robust multichip average (RMA) algorithm to generate expression values.

Quantitative real-time PCR (qRT-PCR) may also be used to measure the differential expression of a plurality of biomarkers. In qRT-PCR, the RNA template is generally reverse transcribed into cDNA, which is then amplified via a PCR reaction. The amount of PCR product is followed cycle-by-cycle in real time, which allows for determination of the initial concentrations of MiRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified. A non-limiting example of a fluorescent reporter probe is a TaqMan® probe (Applied Biosystems, Foster City, Calif.). The fluorescent reporter probe fluoresces when the quencher is removed during the PCR extension cycle. Muliplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, QRT-PCR is typically performed using a reference standard. The ideal reference standard is expressed at a constant level among different tissues, and is unaffected by the experimental treatment. Suitable reference standards include, but are not limited to, MiRNAs for the housekeeping genes glyceraldehyde-3-phosphate-dehydrogenase (GAPDH) and beta-actin. The level of MiRNA in the original sample or the fold change in expression of each biomarker may be determined using calculations well known in the art.

Immunohistochemical staining may also be used to measure the differential expression of a plurality of biomarkers. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye. The labeled biomarker proteins are incubated with the antibody microarray. After washes to remove the unbound proteins, the microarray is scanned. The raw fluorescent intensity data maybe converted into expression values using means known in the art.

Luminex multiplexing microspheres may also be used to measure the differential expression of a plurality of biomarkers. These microscopic polystyrene beads are internally color-coded with fluorescent dyes, such that each bead has a unique spectral signature (of which there are up to 100). Beads with the same signature are tagged with a specific oligonucleotide or specific antibody that will bind the target of interest (i.e., biomarker MiRNA or protein, respectively). The target, in turn, is also tagged with a fluorescent reporter. Hence, there are two sources of color, one from the bead and the other from the reporter molecule on the target. The beads are then incubated with the sample containing the targets, of which up to 100 may be detected in one well. The small size/surface area of the beads and the three dimensional exposure of the beads to the targets allows for nearly solution-phase kinetics during the binding reaction. The captured targets are detected by high-tech fluidics based upon flow cytometry in which lasers excite the internal dyes that identify each bead and also any reporter dye captured during the assay. The data from the acquisition files may be converted into expression values using means known in the art.

In situ hybridization may also be used to measure the differential expression of a plurality of biomarkers. This method permits the localization of MiRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an MiRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple MiRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

The number of biomarkers whose expression is measured in a sample of cells from a subject with cancer may vary. Since the predicted score of survival is based upon the differential expression of the biomarkers, a higher degree of accuracy should be attained when the expression of more biomarkers is measured.

Obtaining a Sample of Cells from a Subject with Cancer

The expression of a plurality of biomarkers will be measured in a sample of cells from a subject with cancer. The type and classification of the cancer can and will vary. The cancer may be an early stage cancer, i.e., stage I or stage II, or it may be a late stage cancer, i.e., stage III or stage IV.

Generally, the sample of cells or tissue sample will be obtained from the subject with cancer by biopsy or surgical resection. The type of biopsy can and will vary, depending upon the location and nature of the cancer. A sample of cells, tissue, or fluid may be removed by needle aspiration biopsy. For this, a fine needle attached to a syringe is inserted through the skin and into the organ or tissue of interest. The needle is typically guided to the region of interest using ultrasound or computed tomography imaging. Once the needle is inserted into the tissue, a vacuum is created with the syringe such that cells or fluid may be sucked through the needle and collected in the syringe. A sample of cells or tissue may also be removed by incisional or core biopsy. For this, a cone, a cylinder, or a tiny bit of tissue is removed from the region of interest. Computed tomography imaging, ultrasound, or an endoscope is generally used to guide this type of biopsy. Lastly, the entire cancerous lesion may be removed by excisional biopsy or surgical resection.

Once a sample of cells or sample of tissue is removed from the subject with cancer, it may be processed for the isolation of RNA or protein using techniques well known in the art and disclosed in standard molecular biology reference books, such as Ausubel et al., (2003) Current Protocols in Molecular Biology, John Wiley & Sons, New York, N.Y. A sample of tissue may also be stored or flash frozen and stored at −80° C. for later use. The biopsied tissue sample may also be fixed with a fixative, such as formaldehyde, paraformaldehyde, or acetic acid/ethanol. The fixed tissue sample may be embedded in wax (paraffin) or a plastic resin. The embedded tissue sample (or frozen tissue sample) may be cut into thin sections. RNA or protein may also be extracted from a fixed or wax-embedded tissue sample.

The subject with cancer will generally be a mammalian subject. Mammals may include primates, livestock animals, and companion animals. Non-limiting examples include: Primates may include humans, apes, monkeys, and gibbons; Livestock animals may include horses, cows, goats, sheep, deer and pigs; Companion animals may include dogs, cats, rabbits, and rodents (including mice, rats, and guinea pigs). In an exemplary embodiment, the subject is a human.

Generating a Risk Score

In certain embodiments, the biomarkers of this invention are related to cancer survival. The differential patterns of expression of a plurality of these biomarkers may be used to predict the survival outcome of a subject with cancer. Certain biomarkers tend to be over-expressed in long-term survivors, whereas other biomarkers tend to be over-expressed in short-term survivors. The unique pattern of expression of a plurality of biomarkers in a subject (i.e., the expression signature) may be used to generate a risk score of survival. Subjects with a high risk score may have a short survival time (<2 years) after surgical resection. Subjects with a low risk score may have a longer survival time (>5 years) after resection.

Regardless of the technique used to measure the differential expression of a plurality of biomarkers, the expression of each biomarker typically will be converted into an expression value. These expression values then will be used to calculate a risk score of survival for a subject with cancer using statistical methods well known in the art. The risk scores may also be calculated using a univariate Cox regression analysis. In one preferred embodiment, the risk scores may be calculated using a partial Cox regression analysis.

The scores generated by a partial Cox regression analysis fall into two groups: 1) those having a positive value; and 2) those having a negative value. A risk score having a positive value is associated with a short survival time, and a risk score having a negative value is associated with a long survival time.

In one embodiment of this method, a tissue sample may be removed by surgical resection from a subject with an early stage cancer. The sample of tissue may be stored in RNAlater or flash frozen, such that RNA may be isolated at a later date. The RNA may be used as a template for qRT-PCR in which the expression of a plurality of biomarkers is analyzed, and the expression data are used to derive a risk score using the partial Cox regression classification method. The risk score may be used to predict whether the subject will be a short-term or a long-term cancer survivor.

In an especially preferred embodiment of this method, a sample of tissue may be collected from a subject with an early stage cancer. RNA may be isolated from the tissue and used to generate labeled probes for a nucleic acid microarray analysis. The expression values generated from the microarray analysis may be used to derive a risk score using the partial Cox regression classification method. The risk score may be used to predict whether the subject will be a short-term or a long-term cancer survivor.

Method for Determining the Prognosis of a Subject with Disease

Another aspect of the invention provides a method for determining the prognosis of a subject with a cancer. The method comprises measuring the differential expression of one or more biomarkers in a sample of cells from the subject. The differential expression of each biomarker is converted into an expression value, and the expression values are used to derive a score for that subject using a statistical method, as detailed above. A score having a positive value is indicative of a poor prognosis or a poor outcome, whereas a score having a negative value is indicative of a good prognosis or a good outcome.

In one embodiment of this method, an expression signature for a subject with an early stage cancer is generated by nucleic acid microarray analysis, and the expression values are used to calculate a score. The calculated score may be used to predict whether the subject will have a good prognosis or a poor prognosis of cancer outcome.

Method for Selecting a Treatment for a Subject with Cancer

A further aspect of the invention provides a method for selecting an effective treatment for a subject with cancer. Once a risk score has been calculated for a subject, that information may be used to decide upon an appropriate course of treatment for the subject. A subject having a positive risk score (i.e., short survival time or poor prognosis) may benefit from an aggressive therapeutic regime. An aggressive therapeutic regime may comprise the appropriate chemotherapy agent or agents. An aggressive therapeutic regime may also comprise radiation therapy. The treatment regime can and will vary, depending upon the type and stage of cancer. A subject having a negative risk score (i.e., long survival time or good prognosis) may not need additional treatment, since the subject is not likely to develop a recurrent cancer.

The cells are maintained under conditions in which the one or more agents inhibits expression or activity of the microRNAs, inhibits expression of one or more target genes of the microRNAs, or inhibits a combination thereof, thereby inhibiting proliferation of the cell.

Methods of identifying an agent that can be used to inhibit proliferation of a cancer cell are also provided. The method comprises contacting one or more microRNAs with an agent to be assessed; contacting one or more target genes with an agent to be assessed; or contacting a combination thereof. If expression of the microRNAs is inhibited in the presence of the agent; of if expression of the target genes is enhanced in the presence of the agent, or a combination thereof occurs in the presence of the agent, then the agent can be used to inhibit proliferation of a follicular thyroid carcinoma cell.

Method of Identifying Therapeutic Agents

Also provided herein are methods of identifying an agent that can be used to treat a patient in need thereof. The method comprises contacting one or more microRNAs with an agent to be assessed; contacting one or more target genes of one or more microRNAs; or contacting a combination thereof. If expression of the microRNAs is inhibited in the presence of the agent; of if expression of the target genes is enhanced in the presence of the agent, or a combination thereof occurs in the presence of the agent, then the agent can be used to inhibit proliferation of a follicular thyroid carcinoma cell.

Agents that can be assessed in the methods provided herein include miRNA inhibitors. Other examples of such agents include pharmaceutical agents, drugs, chemical compounds, ionic compounds, organic compounds, organic ligands, including cofactors, saccharides, recombinant and synthetic peptides, proteins, peptoids, nucleic acid sequences, including genes, nucleic acid products, and antibodies and antigen binding fragments thereof. Such agents can be individually screened or one or more compound(s) can be tested simultaneously in accordance with the methods herein. Large combinatorial libraries of compounds (e.g., organic compounds, recombinant or synthetic peptides, peptoids, nucleic acids) produced by combinatorial chemical synthesis or other methods can be tested. Where compounds selected from a combinatorial library carry unique tags, identification of individual compounds by chromatographic methods is possible. Chemical libraries, microbial broths and phage display libraries can also be tested (screened) in accordance with the methods herein.

Kit for Predicting Survival or Prognosis of a Subject

A further aspect of the invention provides kits for predicting survival or prognosis of a subject with cancer. A kit comprises a plurality of agents for measuring the differential expression of one or more biomarkers, means for converting the expression data into expression values, and means for analyzing the expression values to generate scores that predict survival or prognosis. The agents in the kit for measuring biomarker expression may comprise an array of polynucleotides complementary to the MiRNAs of the biomarkers. In another embodiment, the agents in the kit for measuring biomarker expression may comprise a plurality of PCR probes and/or primers for qRT-PCR.

The invention is also directed to kits for detecting a cancer in an individual comprising one or more reagents for detecting 1) one or more microRNAs; 2) one or more target genes of one or more microRNAs; 3) one or more polypeptides expressed by the target genes or 4) a combination thereof. For example, the kit can comprise hybridization probes, restriction enzymes (e.g., for RFLP analysis), allele-specific oligonucleotides, and antibodies that bind to the polypeptide expressed by the target gene.

In a particular embodiment, the kit comprises at least contiguous nucleotide sequence that is substantially or completely complementary to a region of one or more of the microRNAs. In one embodiment, one or reagents in the kit are labeled, and thus, the kits can further comprise agents capable of detecting the label. The kit can further comprise instructions for detecting a cancer using the components of the kit.

Nucleic Acid Array

Another aspect of the invention provides for a nucleic acid array comprising polynucleotides that hybridize to the MiRNAs of biomarkers of the invention. Generally speaking, the nucleic acid array is comprised of a substrate having at least one address. Nucleic acid arrays are commonly known in the art, and moreover, substrates that comprise nucleic acid arrays are also well known in the art. Non-limiting examples of substrate materials include glass and plastic. A substrate may be shaped like a slide or a chip (i.e. a quadrilateral shape), or alternatively, a substrate may be shaped like a well.

The array of the present invention is comprised of at least one address, wherein the address has disposed thereon a nucleic acid that can hybridize to the MiRNA of a biomarker of the invention. In one embodiment, the array is comprised of multiple addresses, wherein each address has disposed thereon a nucleic acid that can hybridize to the MiRNA of a biomarker for predicting survival of a subject with a lung cancer. The array may also comprise one or more addresses wherein the address has disposed thereon a control nucleic acid. The control may be an internal control (i.e. a control for the array itself) and/or an external control (i.e. a control for the sample applied to the array). An array typically is comprised from between about 1 to about 10,000 addresses. In one embodiment, the array is comprised from between about 10 to about 8,000 addresses. In another embodiment, the array is comprised of no more than 500 addresses. In an alternative embodiment, the array is comprised of no less than 500 addresses. Methods of using nucleic acid arrays are well known in the art.

Methods of Use

In one aspect, there is provided herein a method of diagnosing whether a subject has, or is at risk for developing the disease being assessed and/or treated, comprising measuring the level of at least one gene product in a test sample from the subject and comparing the level of the gene product in the test sample to the level of a corresponding gene product in a control sample. As used herein, a “subject” can be any mammal that has, or is suspected of having, esophageal cancer and/or Barrett's esophagus. In a particular embodiment, the subject is a human who has, or is suspected of having, such disease.

The level of at least one gene product can be measured in cells of a biological sample obtained from the subject. For example, a tissue sample can be removed from a subject suspected of having such disease by conventional biopsy techniques. In another example, a blood sample can be removed from the subject, and white blood cells can be isolated for DNA extraction by standard techniques. The blood or tissue sample is preferably obtained from the subject prior to initiation of radiotherapy, chemotherapy or other therapeutic treatment. A corresponding control tissue or blood sample can be obtained from unaffected tissues of the subject, from a normal human individual or population of normal individuals, or from cultured cells corresponding to the majority of cells in the subject's sample. The control tissue or blood sample is then processed along with the sample from the subject, so that the levels of gene product produced from a given gene in cells from the subject's sample can be compared to the corresponding gene product levels from cells of the control sample.

An alteration (i.e., an increase or decrease) in the level of a gene product in the sample obtained from the subject, relative to the level of a corresponding gene product in a control sample, is indicative of the presence of such disease in the subject. In one embodiment, the level of the at least one gene product in the test sample is greater than the level of the corresponding gene product in the control sample (i.e., expression of the gene product is “up-regulated”). As used herein, expression of a gene product is “up-regulated” when the amount of gene product in a cell or tissue sample from a subject is greater than the amount of the same gene product in a control cell or tissue sample. In another embodiment, the level of the at least one gene product in the test sample is less than the level of the corresponding gene product in the control sample (i.e., expression of the gene product is “down-regulated”). As used herein, expression of a gene is “down-regulated” when the amount of gene product produced from that gene in a cell or tissue sample from a subject is less than the amount produced from the same gene in a control cell or tissue sample. The relative gene expression in the control and normal samples can be determined with respect to one or more RNA expression standards. The standards can comprise, for example, a zero gene expression level, the gene expression level in a standard cell line, or the average level of gene expression previously obtained for a population of normal human controls.

The level of a gene product in a sample can be measured using any technique that is suitable for detecting RNA expression levels in a biological sample. Suitable techniques for determining RNA expression levels in cells from a biological sample (e.g., Northern blot analysis, RT-PCR, in situ hybridization) are well known to those of skill in the art. In a particular embodiment, the level of at least one gene product is detected using Northern blot analysis. For example, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Suitable probes for Northern blot hybridization of a given gene product can be produced from the nucleic acid sequences of the given gene product. Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are incorporated herein by reference.

For example, the nucleic acid probe can be labeled with, e.g., a radionuclide, such as ³H, ³²P, ³³P, ¹⁴C, or ³⁵S; a heavy metal; or a ligand capable of functioning as a specific binding pair member for a labeled ligand (e.g., biotin, avidin or an antibody), a fluorescent molecule, a chemiluminescent molecule, an enzyme or the like.

Probes can be labeled to high specific activity by either the nick translation method of Rigby et al. (1977), J. Mol. Biol. 113:237-251 or by the random priming method of Fienberg et al. (1983), Anal. Biochem. 132:6-13, the entire disclosures of which are incorporated herein by reference. The latter is the method of choice for synthesizing ³²P-labeled probes of high specific activity from single-stranded DNA or from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of gene transcript levels. Using another approach, gene transcript levels can be quantified by computerized imaging systems, such the Molecular Dynamics 400-B 2D Phosphorimager available from Amersham Biosciences, Piscataway, N.J.

Where radionuclide labeling of DNA or RNA probes is not practical, the random-primer method can be used to incorporate an analogue, for example, the dTTP analogue 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In addition to Northern and other RNA hybridization techniques, determining the levels of RNA transcripts can be accomplished using the technique of in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference. Suitable probes for in situ hybridization of a given gene product can be produced from the nucleic acid sequences.

The relative number of gene transcripts in cells can also be determined by reverse transcription of gene transcripts, followed by amplification of the reverse-transcribed transcripts by polymerase chain reaction (RT-PCR). The levels of gene transcripts can be quantified in comparison with an internal standard, for example, the level of MiRNA from a “housekeeping” gene present in the same sample. A suitable “housekeeping” gene for use as an internal standard includes, e.g., myosin or glyceraldehyde-3-phosphate dehydrogenase (G3PDH). The methods for quantitative RT-PCR and variations thereof are within the skill in the art.

In some instances, it may be desirable to simultaneously determine the expression level of a plurality of different gene products in a sample. In other instances, it may be desirable to determine the expression level of the transcripts of all known genes correlated with a cancer. Assessing cancer-specific expression levels for hundreds of genes is time consuming and requires a large amount of total RNA (at least 20 μg for each Northern blot) and autoradiographic techniques that require radioactive isotopes.

To overcome these limitations, an oligolibrary, in microchip format (i.e., a microarray), may be constructed containing a set of probe oligodeoxynucleotides that are specific for a set of genes or gene products. Using such a microarray, the expression level of multiple microRNAs in a biological sample can be determined by reverse transcribing the RNAs to generate a set of target oligodeoxynucleotides, and hybridizing them to probe oligodeoxynucleotides on the microarray to generate a hybridization, or expression, profile. The hybridization profile of the test sample can then be compared to that of a control sample to determine which microRNAs have an altered expression level in such disease. As used herein, “probe oligonucleotide” or “probe oligodeoxynucleotide” refers to an oligonucleotide that is capable of hybridizing to a target oligonucleotide. “Target oligonucleotide” or “target oligodeoxynucleotide” refers to a molecule to be detected (e.g., via hybridization). By “specific probe oligonucleotide” or “probe oligonucleotide specific for a gene product” is meant a probe oligonucleotide that has a sequence selected to hybridize to a specific gene product, or to a reverse transcript of the specific gene product.

An “expression profile” or “hybridization profile” of a particular sample is essentially a fingerprint of the state of the sample; while two states may have any particular gene similarly expressed, the evaluation of a number of genes simultaneously allows the generation of a gene expression profile that is unique to the state of the cell. That is, normal cells may be distinguished from the cells, and within the cells, different prognosis states (good or poor long term survival prospects, for example) may be determined. By comparing expression profiles of the cells in different states, information regarding which genes are important (including both up- and down-regulation of genes) in each of these states is obtained. The identification of sequences that are differentially expressed in the cells or normal cells, as well as differential expression resulting in different prognostic outcomes, allows the use of this information in a number of ways. For example, a particular treatment regime may be evaluated (e.g., to determine whether a chemotherapeutic drug act to improve the long-term prognosis in a particular patient). Similarly, diagnosis may be done or confirmed by comparing patient samples with the known expression profiles. Furthermore, these gene expression profiles (or individual genes) allow screening of drug candidates that suppress the expression profile or convert a poor prognosis profile to a better prognosis profile.

Accordingly, the invention provides methods of diagnosing whether a subject has, or is at risk for developing, such disease, comprising reverse transcribing RNA from a test sample obtained from the subject to provide a set of target oligo-deoxynucleotides, hybridizing the target oligo-deoxynucleotides to a microarray comprising miRNA-specific probe oligonucleotides to provide a hybridization profile for the test sample, and comparing the test sample hybridization profile to a hybridization profile generated from a control sample, wherein an alteration in the signal of at least one miRNA is indicative of the subject either having, or being at risk for developing, such disease.

The invention also provides methods of diagnosing such disease associated with one or more prognostic markers, comprising measuring the level of at least one gene product in a test sample from a subject and comparing the level of the at least one gene product in the test sample to the level of a corresponding gene product in a control sample. An alteration (e.g., an increase, a decrease) in the signal of at least one gene product in the test sample relative to the control sample is indicative of the subject either having, or being at risk for developing, such disease associated with the one or more prognostic markers.

The disease can be associated with one or more prognostic markers or features, including, a marker associated with an adverse (i.e., negative) prognosis, or a marker associated with a good (i.e., positive) prognosis. In certain embodiments, such disease that is diagnosed using the methods described herein is associated with one or more adverse prognostic features.

Particular microRNAs whose expression is altered in the cells associated with each of these prognostic markers are described herein. In one embodiment, the level of the at least one gene product is measured by reverse transcribing RNA from a test sample obtained from the subject to provide a set of target oligodeoxynucleotides, hybridizing the target oligodeoxynucleotides to a microarray that comprises miRNA-specific probe oligonucleotides to provide a hybridization profile for the test sample, and comparing the test sample hybridization profile to a hybridization profile generated from a control sample.

Without wishing to be bound by any one theory, it is believed that alterations in the level of one or more gene products in cells can result in the deregulation of one or more intended targets for these gene products, which can lead to the formation of such disease. Therefore, altering the level of the gene product (e.g., by decreasing the level of a gene product that is up-regulated in the cells, by increasing the level of a gene product that is down-regulated in cancer cells) may successfully treat such disease. Examples of putative gene targets for gene products that are deregulated in the cells are described herein.

Accordingly, the present invention encompasses methods of treating such disease in a subject, wherein at least one gene product is de-regulated (e.g., down-regulated, up-regulated) in the cancer cells of the subject. When the at least one isolated gene product is down-regulated in the cells, the method comprises administering an effective amount of the at least one isolated gene product such that proliferation of cancer cells in the subject is inhibited. When the at least one isolated gene product is up-regulated in the cancer cells, the method comprises administering to the subject an effective amount of at least one compound for inhibiting expression of the at least one gene, referred to herein as gene expression inhibition compounds, such that proliferation of the cells is inhibited.

The terms “treat”, “treating” and “treatment”, as used herein, refer to ameliorating symptoms associated with a disease or condition, for example, including preventing or delaying the onset of the disease symptoms, and/or lessening the severity or frequency of symptoms of the disease or condition. The terms “subject” and “individual” are defined herein to include animals, such as mammals, including but not limited to, primates, cows, sheep, goats, horses, dogs, cats, rabbits, guinea pigs, rats, mice or other bovine, ovine, equine, canine, feline, rodent, or murine species. In a preferred embodiment, the animal is a human.

As used herein, an “effective amount” of an isolated gene product is an amount sufficient to inhibit proliferation of a cancer cell in a subject suffering from such disease. One skilled in the art can readily determine an effective amount of a gene product to be administered to a given subject, by taking into account factors, such as the size and weight of the subject; the extent of disease penetration; the age, health and sex of the subject; the route of administration; and whether the administration is regional or systemic.

For example, an effective amount of an isolated gene product can be based on the approximate or estimated body weight of a subject to be treated. Preferably, such effective amounts are administered parenterally or enterally, as described herein. For example, an effective amount of the isolated gene product administered to a subject can range from about 5-3000 micrograms/kg of body weight, from about 700-1000 micrograms/kg of body weight, or greater than about 1000 micrograms/kg of body weight.

One skilled in the art can also readily determine an appropriate dosage regimen for the administration of an isolated gene product to a given subject. For example, a gene product can be administered to the subject once (e.g., as a single injection or deposition). Alternatively, a gene product can be administered once or twice daily to a subject for a period of from about three to about twenty-eight days, more particularly from about seven to about ten days. In a particular dosage regimen, a gene product is administered once a day for seven days. Where a dosage regimen comprises multiple administrations, it is understood that the effective amount of the gene product administered to the subject can comprise the total amount of gene product administered over the entire dosage regimen.

As used herein, an “isolated” gene product is one which is synthesized, or altered or removed from the natural state through human intervention. For example, a synthetic gene product, or a gene product partially or completely separated from the coexisting materials of its natural state, is considered to be “isolated.” An isolated gene product can exist in substantially-purified form, or can exist in a cell into which the gene product has been delivered. Thus, a gene product which is deliberately delivered to, or expressed in, a cell is considered an “isolated” gene product. A gene product produced inside a cell from a precursor molecule is also considered to be an “isolated” molecule.

Isolated gene products can be obtained using a number of standard techniques. For example, the gene products can be chemically synthesized or recombinantly produced using methods known in the art. In one embodiment, gene products are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.) and Cruachem (Glasgow, UK).

Alternatively, the gene products can be expressed from recombinant circular or linear DNA plasmids using any suitable promoter. Suitable promoters for expressing RNA from a plasmid include, e.g., the U6 or H1 RNA pol III promoter sequences, or the cytomegalovirus promoters. Selection of other suitable promoters is within the skill in the art. The recombinant plasmids of the invention can also comprise inducible or regulatable promoters for expression of the gene products in cancer cells.

The gene products that are expressed from recombinant plasmids can be isolated from cultured cell expression systems by standard techniques. The gene products which are expressed from recombinant plasmids can also be delivered to, and expressed directly in, the cancer cells. The use of recombinant plasmids to deliver the gene products to cancer cells is discussed in more detail below.

The gene products can be expressed from a separate recombinant plasmid, or they can be expressed from the same recombinant plasmid. In one embodiment, the gene products are expressed as RNA precursor molecules from a single plasmid, and the precursor molecules are processed into the functional gene product by a suitable processing system, including, but not limited to, processing systems extant within a cancer cell. Other suitable processing systems include, e.g., the in vitro Drosophila cell lysate system (e.g., as described in U.S. Published Patent Application No. 2002/0086356 to Tuschl et al., the entire disclosure of which are incorporated herein by reference) and the E. coli RNAse III system (e.g., as described in U.S. Published Patent Application No. 2004/0014113 to Yang et al., the entire disclosure of which are incorporated herein by reference).

Selection of plasmids suitable for expressing the gene products, methods for inserting nucleic acid sequences into the plasmid to express the gene products, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are incorporated herein by reference.

In one embodiment, a plasmid expressing the gene products comprises a sequence encoding a precursor RNA under the control of the CMV intermediate-early promoter. As used herein, “under the control” of a promoter means that the nucleic acid sequences encoding the gene product are located 3′ of the promoter, so that the promoter can initiate transcription of the gene product coding sequences.

The gene products can also be expressed from recombinant viral vectors. It is contemplated that the gene products can be expressed from two separate recombinant viral vectors, or from the same viral vector. The RNA expressed from the recombinant viral vectors can either be isolated from cultured cell expression systems by standard techniques, or can be expressed directly in cancer cells. The use of recombinant viral vectors to deliver the gene products to cancer cells is discussed in more detail below.

The recombinant viral vectors of the invention comprise sequences encoding the gene products and any suitable promoter for expressing the RNA sequences. Suitable promoters include, for example, the U6 or H1 RNA pol III promoter sequences, or the cytomegalovirus promoters. Selection of other suitable promoters is within the skill in the art. The recombinant viral vectors of the invention can also comprise inducible or regulatable promoters for expression of the gene products in a cancer cell.

Any viral vector capable of accepting the coding sequences for the gene products can be used; for example, vectors derived from adenovirus (AV); adeno-associated virus (AAV); retroviruses (e.g., lentiviruses (LV), Rhabdoviruses, murine leukemia virus); herpes virus, and the like. The tropism of the viral vectors can be modified by pseudotyping the vectors with envelope proteins or other surface antigens from other viruses, or by substituting different viral capsid proteins, as appropriate.

For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors that express different capsid protein serotypes are within the skill in the art; see, e.g., Rabinowitz, J. E., et al. (2002), J. Virol. 76:791-801, the entire disclosure of which is incorporated herein by reference.

Selection of recombinant viral vectors suitable for use in the invention, methods for inserting nucleic acid sequences for expressing RNA into the vector, methods of delivering the viral vector to the cells of interest, and recovery of the expressed RNA products are within the skill in the art. See, for example, Dornburg (1995), Gene Therap. 2:301-310; Eglitis (1988), Biotechniques 6:608-614; Miller (1990), Hum. Gene Therap. 1:5-14; and Anderson (1998), Nature 392:25-30, the entire disclosures of which are incorporated herein by reference.

In certain embodiments, suitable viral vectors are those derived from AV and AAV. A suitable AV vector for expressing the gene products, a method for constructing the recombinant AV vector, and a method for delivering the vector into target cells, are described in Xia et al. (2002), Nat. Biotech. 20:1006-1010, the entire disclosure of which is incorporated herein by reference. Suitable AAV vectors for expressing the gene products, methods for constructing the recombinant AAV vector, and methods for delivering the vectors into target cells are described in Samulski et al. (1987), J. Virol. 61:3096-3101; Fisher et al. (1996), J. Virol., 70:520-532; Samulski et al. (1989), J. Virol. 63:3822-3826; U.S. Pat. No. 5,252,479; U.S. Pat. No. 5,139,941; International Patent Application No. WO 94/13788; and International Patent Application No. WO 93/24641, the entire disclosures of which are incorporated herein by reference.

In a certain embodiment, a recombinant AAV viral vector of the invention comprises a nucleic acid sequence encoding a precursor RNA in operable connection with a polyT termination sequence under the control of a human U6 RNA promoter. As used herein, “in operable connection with a polyT termination sequence” means that the nucleic acid sequences encoding the sense or antisense strands are immediately adjacent to the polyT termination signal in the 5′ direction. During transcription of the sequences from the vector, the polyT termination signals act to terminate transcription.

In other embodiments of the treatment methods of the invention, an effective amount of at least one compound which inhibits expression can also be administered to the subject. As used herein, “inhibiting gene expression” means that the production of the active, mature form of gene product after treatment is less than the amount produced prior to treatment. One skilled in the art can readily determine whether expression has been inhibited in a cancer cell, using for example the techniques for determining transcript level discussed above for the diagnostic method. Inhibition can occur at the level of gene expression (i.e., by inhibiting transcription of a gene encoding the gene product) or at the level of processing (e.g., by inhibiting processing of a precursor into a mature, active gene product).

As used herein, an “effective amount” of a compound that inhibits expression is an amount sufficient to inhibit proliferation of a cancer cell in a subject suffering from a cancer associated with a cancer-associated chromosomal feature. One skilled in the art can readily determine an effective amount of an expression-inhibiting compound to be administered to a given subject, by taking into account factors, such as the size and weight of the subject; the extent of disease penetration; the age, health and sex of the subject; the route of administration; and whether the administration is regional or systemic.

For example, an effective amount of the expression-inhibiting compound can be based on the approximate or estimated body weight of a subject to be treated. Such effective amounts are administered parenterally or enterally, among others, as described herein. For example, an effective amount of the expression-inhibiting compound administered to a subject can range from about 5-3000 micrograms/kg of body weight, from about 700-1000 micrograms/kg of body weight, or it can be greater than about 1000 micrograms/kg of body weight.

One skilled in the art can also readily determine an appropriate dosage regimen for administering a compound that inhibits expression to a given subject. For example, an expression-inhibiting compound can be administered to the subject once (e.g., as a single injection or deposition). Alternatively, an expression-inhibiting compound can be administered once or twice daily to a subject for a period of from about three to about twenty-eight days, more preferably from about seven to about ten days. In a particular dosage regimen, an expression-inhibiting compound is administered once a day for seven days. Where a dosage regimen comprises multiple administrations, it is understood that the effective amount of the expression-inhibiting compound administered to the subject can comprise the total amount of compound administered over the entire dosage regimen.

Suitable compounds for inhibiting expression include double-stranded RNA (such as short- or small-interfering RNA or “siRNA”), antisense nucleic acids, and enzymatic RNA molecules, such as ribozymes. Each of these compounds can be targeted to a given gene product and destroy or induce the destruction of the target gene product.

For example, expression of a given gene can be inhibited by inducing RNA interference of the gene with an isolated double-stranded RNA (“dsRNA”) molecule which has at least 90%, for example at least 95%, at least 98%, at least 99% or 100%, sequence homology with at least a portion of the gene product. In a particular embodiment, the dsRNA molecule is a “short or small interfering RNA” or “siRNA.”

siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”). The sense strand comprises a nucleic acid sequence which is substantially identical to a nucleic acid sequence contained within the target gene product.

As used herein, a nucleic acid sequence in an siRNA which is “substantially identical” to a target sequence contained within the target MiRNA is a nucleic acid sequence that is identical to the target sequence, or that differs from the target sequence by one or two nucleotides. The sense and antisense strands of the siRNA can comprise two complementary, single-stranded RNA molecules, or can comprise a single molecule in which two complementary portions are base-paired and are covalently linked by a single-stranded “hairpin” area.

The siRNA can also be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides.

One or both strands of the siRNA can also comprise a 3′ overhang. As used herein, a “3′ overhang” refers to at least one unpaired nucleotide extending from the 3′-end of a duplexed RNA strand. Thus, in certain embodiments, the siRNA comprises at least one 3′ overhang of from 1 to about 6 nucleotides (which includes ribonucleotides or deoxyribonucleotides) in length, from 1 to about 5 nucleotides in length, from 1 to about 4 nucleotides in length, or from about 2 to about 4 nucleotides in length. In a particular embodiment, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above for the isolated gene products. Exemplary methods for producing and testing dsRNA or siRNA molecules are described in U.S. Published Patent Application No. 2002/0173478 to Gewirtz and in U.S. Published Patent Application No. 2004/0018176 to Reich et al., the entire disclosures of which are incorporated herein by reference.

Expression of a given gene can also be inhibited by an antisense nucleic acid. As used herein, an “antisense nucleic acid” refers to a nucleic acid molecule that binds to target RNA by means of RNA-RNA or RNA-DNA or RNA-peptide nucleic acid interactions, which alters the activity of the target RNA. Antisense nucleic acids suitable for use in the present methods are single-stranded nucleic acids (e.g., RNA, DNA, RNA-DNA chimeras, PNA) that generally comprise a nucleic acid sequence complementary to a contiguous nucleic acid sequence in a gene product. The antisense nucleic acid can comprise a nucleic acid sequence that is 50-100% complementary, 75-100% complementary, or 95-100% complementary to a contiguous nucleic acid sequence in a gene product. Nucleic acid sequences for the gene products are provided herein. Without wishing to be bound by any theory, it is believed that the antisense nucleic acids activate RNase H or another cellular nuclease that digests the gene product/antisense nucleic acid duplex.

Antisense nucleic acids can also contain modifications to the nucleic acid backbone or to the sugar and base moieties (or their equivalent) to enhance target specificity, nuclease resistance, delivery or other properties related to efficacy of the molecule. Such modifications include cholesterol moieties, duplex intercalators, such as acridine, or one or more nuclease-resistant groups.

Antisense nucleic acids can be produced chemically or biologically, or can be expressed from a recombinant plasmid or viral vector, as described above for the isolated gene products. Exemplary methods for producing and testing are within the skill in the art; see, e.g., Stein and Cheng (1993), Science 261:1004 and U.S. Pat. No. 5,849,902 to Woolf et al., the entire disclosures of which are incorporated herein by reference.

Expression of a given gene can also be inhibited by an enzymatic nucleic acid. As used herein, an “enzymatic nucleic acid” refers to a nucleic acid comprising a substrate binding region that has complementarity to a contiguous nucleic acid sequence of a gene product, and which is able to specifically cleave the gene product. The enzymatic nucleic acid substrate binding region can be, for example, 50-100% complementary, 75-100% complementary, or 95-100% complementary to a contiguous nucleic acid sequence in a gene product. The enzymatic nucleic acids can also comprise modifications at the base, sugar, and/or phosphate groups. An exemplary enzymatic nucleic acid for use in the present methods is a ribozyme.

The enzymatic nucleic acids can be produced chemically or biologically, or can be expressed from a recombinant plasmid or viral vector, as described above for the isolated gene products. Exemplary methods for producing and testing dsRNA or siRNA molecules are described in Werner and Uhlenbeck (1995), Nucl. Acids Res. 23:2092-96; Hammann et al. (1999), Antisense and Nucleic Acid Drug Dev. 9:25-31; and U.S. Pat. No. 4,987,071 to Cech et al, the entire disclosures of which are incorporated herein by reference.

Administration of at least one gene product, or at least one compound for inhibiting expression, will inhibit the proliferation of cancer cells in a subject who has a cancer associated with a cancer-associated chromosomal feature. As used herein, to “inhibit the proliferation of a cancer cell” means to kill the cell, or permanently or temporarily arrest or slow the growth of the cell. Inhibition of cancer cell proliferation can be inferred if the number of such cells in the subject remains constant or decreases after administration of the gene products or gene expression-inhibiting compounds. An inhibition of cancer cell proliferation can also be inferred if the absolute number of such cells increases, but the rate of tumor growth decreases.

The number of cancer cells in a subject's body can be determined by direct measurement, or by estimation from the size of primary or metastatic tumor masses. For example, the number of cancer cells in a subject can be measured by immunohistological methods, flow cytometry, or other techniques designed to detect characteristic surface markers of cancer cells.

The gene products or gene expression-inhibiting compounds can be administered to a subject by any means suitable for delivering these compounds to cancer cells of the subject. For example, the gene products or expression inhibiting compounds can be administered by methods suitable to transfect cells of the subject with these compounds, or with nucleic acids comprising sequences encoding these compounds.

A gene product or gene expression inhibiting compound can also be administered to a subject by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition, including subcutaneous infusion (such as by osmotic pumps); direct application to the tissue of interest, for example by a catheter or other placement device (e.g., a retinal pellet or a suppository or an implant comprising a porous, non-porous, or gelatinous material); and inhalation. Particularly suitable administration routes are injection, infusion and intravenous administration into the patient.

In the present methods, a gene product or gene product expression inhibiting compound can be administered to the subject either as naked RNA, in combination with a delivery reagent, or as a nucleic acid (e.g., a recombinant plasmid or viral vector) comprising sequences that express the gene product or expression inhibiting compound. Suitable delivery reagents include, e.g., the Mirus Transit TKO lipophilic reagent; lipofectin; lipofectamine; cellfectin; polycations (e.g., polylysine), and liposomes.

Recombinant plasmids and viral vectors comprising sequences that express the gene products or gene expression inhibiting compounds, and techniques for delivering such plasmids and vectors to cancer cells, are discussed herein.

In a particular embodiment, liposomes are used to deliver a gene product or gene expression-inhibiting compound (or nucleic acids comprising sequences encoding them) to a subject. Liposomes can also increase the blood half-life of the gene products or nucleic acids. Suitable liposomes for use in the invention can be formed from standard vesicle-forming lipids, which generally include neutral or negatively charged phospholipids and a sterol, such as cholesterol. The selection of lipids is generally guided by consideration of factors, such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are incorporated herein by reference.

The liposomes for use in the present methods can comprise a ligand molecule that targets the liposome to cancer cells. Ligands which bind to receptors prevalent in cancer cells, such as monoclonal antibodies that bind to tumor cell antigens, are preferred.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In a particularly preferred embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is incorporated herein by reference.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers, such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example solid tumors, will efficiently accumulate these liposomes; see Gabizon, et al. (1988), Proc. Natl. Acad. Sci., U.S.A., 18:6949-53. In addition, the reduced uptake by the RES lowers the toxicity of stealth liposomes by preventing significant accumulation of the liposomes in the liver and spleen. Thus, liposomes that are modified with opsonization-inhibition moieties are particularly suited to deliver the gene products or gene expression inhibition compounds (or nucleic acids comprising sequences encoding them) to tumor cells.

The gene products or gene expression inhibition compounds can be formulated as pharmaceutical compositions, sometimes called “medicaments,” prior to administering them to a subject, according to techniques known in the art. Accordingly, the invention encompasses pharmaceutical compositions for treating ALL. In one embodiment, the pharmaceutical compositions comprise at least one isolated gene product and a pharmaceutically-acceptable carrier. In a particular embodiment, the at least one gene product corresponds to a gene product that has a decreased level of expression in ALL cells relative to suitable control cells.

In other embodiments, the pharmaceutical compositions of the invention comprise at least one expression inhibition compound. In a particular embodiment, the at least one gene expression inhibition compound is specific for a gene whose expression is greater in ALL cells than control cells.

Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is incorporated herein by reference.

The present pharmaceutical formulations comprise at least one gene product or gene expression inhibition compound (or at least one nucleic acid comprising sequences encoding them) (e.g., 0.1 to 90% by weight), or a physiologically acceptable salt thereof, mixed with a pharmaceutically-acceptable carrier. The pharmaceutical formulations of the invention can also comprise at least one gene product or gene expression inhibition compound (or at least one nucleic acid comprising sequences encoding them) which are encapsulated by liposomes and a pharmaceutically-acceptable carrier.

Especially suitable pharmaceutically-acceptable carriers are water, buffered water, normal saline, 0.4% saline, 0.3% glycine, hyaluronic acid and the like.

In a particular embodiment, the pharmaceutical compositions of the invention comprise at least one gene product or gene expression inhibition compound (or at least one nucleic acid comprising sequences encoding them) which is resistant to degradation by nucleases. One skilled in the art can readily synthesize nucleic acids which are nuclease resistant, for example by incorporating one or more ribonucleotides that are modified at the 2′-position into the gene products. Suitable 2′-modified ribonucleotides include those modified at the 2′-position with fluoro, amino, alkyl, alkoxy, and O-allyl.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid pharmaceutical compositions of the invention, conventional nontoxic solid pharmaceutically-acceptable carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of the at least one gene product or gene expression inhibition compound (or at least one nucleic acid comprising sequences encoding them). A pharmaceutical composition for aerosol (inhalational) administration can comprise 0.01-20% by weight, preferably 1%-10% by weight, of the at least one gene product or gene expression inhibition compound (or at least one nucleic acid comprising sequences encoding them) encapsulated in a liposome as described above, and a propellant. A carrier can also be included as desired; e.g., lecithin for intranasal delivery.

The invention also encompasses methods of identifying an anti-cancer agent, comprising providing a test agent to a cell and measuring the level of at least one gene product in the cell. In one embodiment, the method comprises providing a test agent to a cell and measuring the level of at least one gene product associated with decreased expression levels in the cells. An increase in the level of the gene product in the cell, relative to a suitable control cell, is indicative of the test agent being an anti-cancer agent.

In other embodiments the method comprises providing a test agent to a cell and measuring the level of at least one gene product associated with increased expression levels in the cells. A decrease in the level of the gene product in the cell, relative to a suitable control cell, is indicative of the test agent being an anti-cancer agent.

Suitable agents include, but are not limited to drugs (e.g., small molecules, peptides), and biological macromolecules (e.g., proteins, nucleic acids). The agent can be produced recombinantly, synthetically, or it may be isolated (i.e., purified) from a natural source. Various methods for providing such agents to a cell (e.g., transfection) are well known in the art, and several of such methods are described hereinabove. Methods for detecting the expression of at least one gene product (e.g., Northern blotting, in situ hybridization, RT-PCR, expression profiling) are also well known in the art.

### DEFINITIONS

The term “array” is used interchangeably with the term “microarray” herein.

The term “cancer,” as used herein, refers to the physiological condition in mammals that is typically characterized by unregulated cell proliferation, and the ability of those cells to invade other tissues.

The term “expression,” as used herein, refers to the conversion of the DNA sequence information into messenger RNA (MiRNA) or protein. Expression may be monitored by measuring the levels of full-length MiRNA, MiRNA fragments, full-length protein, or protein fragments.

The term “fusion protein” is intended to describe at least two polypeptides, typically from different sources, which are operably linked. With regard to polypeptides, the term operably linked is intended to mean that the two polypeptides are connected in a manner such that each polypeptide can serve its intended function. Typically, the two polypeptides are covalently attached through peptide bonds. The fusion protein is preferably produced by standard recombinant DNA techniques. For example, a DNA molecule encoding the first polypeptide is ligated to another DNA molecule encoding the second polypeptide, and the resultant hybrid DNA molecule is expressed in a host cell to produce the fusion protein. The DNA molecules are ligated to each other in a 5′ to 3′ orientation such that, after ligation, the translational frame of the encoded polypeptides is not altered (i.e., the DNA molecules are ligated to each other in-frame).

The phrase “gene expression signature,” as used herein refers to the unique pattern of gene expression in a cell, and in particular, a cancer cell.

The term “hybridization,” as used herein, refers to the process of binding, annealing, or base-pairing between two single-stranded nucleic acids. The “stringency of hybridization” is determined by the conditions of temperature and ionic strength. Nucleic acid hybrid stability is expressed as the melting temperature or Tm, which is the temperature at which the hybrid is 50% denatured under defined conditions. Equations have been derived to estimate the Tm of a given hybrid; the equations take into account the G+C content of the nucleic acid, the length of the hybridization probe, etc. (e.g., Sambrook et al., 1989). To maximize the rate of annealing of the probe with its target, hybridizations are generally carried out in solutions of high ionic strength (6×SSC or 6×SSPE) at a temperature that is about 2025° C. below the Tm. If the sequences to be hybridized are not identical, then the hybridization temperature is reduced 1-1.5° C. for every 1% of mismatch. In general, the washing conditions should be as stringent as possible (i.e., low ionic strength at a temperature about 12-20° C. below the calculated Tm). As an example, highly stringent conditions typically involve hybridizing at 68° C. in 6×SSC/5×Denhardt's solution/1.0% SDS and washing in 0.2×SSC/0.1% SDS at 65° C. The optimal hybridization conditions generally differ between hybridizations performed in solution and hybridizations using immobilized nucleic acids. One skilled in the art will appreciate which parameters to manipulate to optimize hybridization.

The term “nucleic acid,” as used herein, refers to sequences of linked nucleotides. The nucleotides may be deoxyribonucleotides or ribonucleotides, they may be standard or non-standard nucleotides; they may be modified or derivatized nucleotides; they may be synthetic analogs. The nucleotides may be linked by phosphodiester bonds or non-hydrolyzable bonds. The nucleic acid may comprise a few nucleotides (i.e., oligonucleotide), or it may comprise many nucleotides (i.e., polynucleotide). The nucleic acid may be single-stranded or double-stranded.

The term “prognosis,” as used herein refers to the probable course and outcome of a cancer, and in particular, the likelihood of recovery.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

### REFERENCES

The publication and other material used herein to illuminate the invention or provide additional details respecting the practice of the invention, are incorporated by reference herein, and for convenience are provided in the following bibliography.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.
1. Parkin, D M, Bray, F, Ferlay, J, Pisani, P (2005) Global cancer statistics, 2002. CA Cancer J Clin 55:74-108.
2. Sant, M et al. (2003) EUROCARE-3: survival of cancer patients diagnosed 1990-94-results and commentary. Ann. Oncol. 14 Suppl 5:v61-118.
3. (2007) Surveillance and Epidemiology and End Results (SEER).
4. Crew, K D, Neugut, A I (2004) Epidemiology of upper gastrointestinal malignancies. Semin. Oncol. 31:450-464.
5. Crew, K D, Neugut, A I (2004) Epidemiology of upper gastrointestinal malignancies. Semin. Oncol. 31:450-464.
6. Cameron, A J, Ott, B J, Payne, W S (1985) The incidence of adenocarcinoma in columnar-lined (Barrett's) esophagus. N. Engl. J. Med. 313:857-859.
7. Maley, C C, Rustgi, A K (2006) Barrett's esophagus and its progression to adenocarcinoma. J. Natl. Compr. Canc. Netw. 4:367-374.
8. Lagos-Quintana, M, Rauhut, R, Lendeckel, W, Tuschl, T (2001) Identification of novel genes coding for small expressed RNAs. Science 294:853-858.
9. Lau, N C, Lim, L P, Weinstein, E G, Bartel, D P (2001) An abundant class of tiny RNAs with probable regulatory roles in Caenorhabditis elegans. Science 294:858-862.
10. Lee, R C, Ambros, V (2001) An extensive class of small RNAs in Caenorhabditis elegans. Science 294:862-864.
11. Lee, R C, Feinbaum, R L, Ambros, V (1993) The C. elegans heterochronic gene lin-4 encodes small RNAs with antisense complementarity to lin-14. Cell 75:843-854.
12. Griffiths-Jones, S et al. (2006) miRBase: microRNA sequences, targets and gene nomenclature. Nucleic Acids Res 34:D140-D144.
13. Griffiths-Jones, S (2004) The microRNA Registry. Nucleic Acids Res. 32:D109-D111.
14. Bartel, D P (2004) MicroRNAs: genomics, biogenesis, mechanism, and function. Cell 116:281-297.
15. Esquela-Kerscher, A, Slack, F J (2006) Oncomirs—microRNAs with a role in cancer. Nat. Rev. Cancer 6:259-269.
16. Volinia, S et al. (2006) A microRNA expression signature of human solid tumors defines cancer gene targets. Proc. Natl. Acad. Sci U.S.A 103:2257-2261.
17. Lu, J et al. (2005) MicroRNA expression profiles classify human cancers. Nature 435:834-838.
18. Sevignani, C, Calin, G A, Siracusa, L D, Croce, C M (2006) Mammalian microRNAs: a small world for fine-tuning gene expression. Mamm. Genome 17:189-202.
19. Calin, G A et al. (2004) Human microRNA genes are frequently located at fragile sites and genomic regions involved in cancers. Proc. Natl. Acad. Sci. U.S.A 101:2999-3004.
20. Yanaihara, N et al. (2006) Unique microRNA molecular profiles in lung cancer diagnosis and prognosis. Cancer Cell 9:189-198.
21. Esquela-Kerscher, A et al. (2008) The let-7 microRNA reduces tumor growth in mouse models of lung cancer. Cell Cycle 7:759-764.
22. Johnson, S M et al. (2005) RAS is regulated by the let-7 microRNA family. Cell 120:635-647.
23. Takamizawa, J et al. (2004) Reduced expression of the let-7 microRNAs in human lung cancers in association with shortened postoperative survival. Cancer Res. 64:3753-3756.
24. Schetter, A J et al. (2008) MicroRNA expression profiles associated with prognosis and therapeutic outcome in colon adenocarcinoma. JAMA 299:425-436.
25. Budhu, A et al. (2008) Identification of metastasis-related microRNAs in hepatocellular carcinoma. Hepatology 47:897-907.
26. Lee, E J et al. (2007) Expression profiling identifies microRNA signature in pancreatic cancer. Int. J. Cancer 120:1046-1054.
27. Iorio, M V et al. (2005) MicroRNA gene expression deregulation in human breast cancer. Cancer Res. 65:7065-7070.
28. He, H et al. (2005) The role of microRNA genes in papillary thyroid carcinoma. Proc. Natl. Acad. Sci. U.S.A 102:19075-19080.
29. Krutzfeldt, J et al. (2005) Silencing of microRNAs in vivo with ‘antagomirs’. Nature 438:685-689.
30. Elmen, J et al. (2008) LNA-mediated microRNA silencing in non-human primates. Nature
31. Sugito, N et al. (2006) RNASEN regulates cell proliferation and affects survival in esophageal cancer patients. Clin Cancer Res 12:7322-7328.
32. Feber, A et al. (2008) MicroRNA expression profiles of esophageal cancer. J. Thorac. Cardiovasc. Surg. 135:255-260.
33. Watson, D I et al. (2007) Hp24 microrna expression profiles in barrett's oesophagus. ANZ. J. Surg. 77 Suppl 1:A45.
34. Watson, D I et al. (2007) Hp24 microrna expression profiles in barrett's oesophagus. ANZ. J. Surg. 77 Suppl 1:A45.
35. Liu, C G et al. (2004) An oligonucleotide microchip for genome-wide microRNA profiling in human and mouse tissues. Proc Natl Acad Sci U.S.A 101:9740-9744.
36. Calin, G A et al. (2004) Human microRNA genes are frequently located at fragile sites and genomic regions involved in cancers. Proc. Natl. Acad. Sci. U.S.A 101:2999-3004.
37. Esquela-Kerscher, A et al. (2008) The let-7 microRNA reduces tumor growth in mouse models of lung cancer. Cell Cycle 7:759-764.
38. Kumar, M S et al. (2008) Suppression of non-small cell lung tumor development by the let-7 microRNA family. Proc. Natl. Acad. Sci. U.S.A 105:3903-3908.
39. Takamizawa, J et al. (2004) Reduced expression of the let-7 microRNAs in human lung cancers in association with shortened postoperative survival. Cancer Res. 64:3753-3756.
40. Johnson, S M et al. (2005) RAS is regulated by the let-7 microRNA family. Cell 120:635-647.
41. Yanaihara, N et al. (2006) Unique microRNA molecular profiles in lung cancer diagnosis and prognosis. Cancer Cell 9:189-198.
42. Volinia, S et al. (2006) A microRNA expression signature of human solid tumors defines cancer gene targets. Proc. Natl. Acad. Sci U.S.A 103:2257-2261.
43. Iorio, M V et al. (2005) MicroRNA gene expression deregulation in human breast cancer. Cancer Res 65:7065-7070.
44. Si, M L et al. (2007) miR-21-mediated tumor growth. Oncogene 26:2799-2803.
45. Lee, E J et al. (2007) Expression profiling identifies microRNA signature in pancreatic cancer. Int. J Cancer 120:1046-1054.
46. Fulci, V et al. (2007) Quantitative technologies establish a novel microRNA profile of chronic lymphocytic leukemia. Blood 109:4944-4951.
47. Metzler, M et al. (2004) High expression of precursor microRNA-155/BIC RNA in children with Burkitt lymphoma. Genes Chromosomes. Cancer 39:167-169.
48. Eis, P S et al. (2005) Accumulation of miR-155 and BIC RNA in human B cell lymphomas. Proc. Natl. Acad. Sci. U.S.A 102:3627-3632.
49. O'Connell, R M et al. (2007) MicroRNA-155 is induced during the macrophage inflammatory response. Proc. Natl. Acad. Sci U.S.A 104:1604-1609.
50. Meng, F et al. (2007) MicroRNA-21 regulates expression of the PTEN tumor suppressor gene in human hepatocellular cancer. Gastroenterology 133:647-658.
51. Zhu, S, Si, M L, Wu, H, Mo, Y Y (2007) MicroRNA-21 targets the tumor suppressor gene tropomyosin 1 (TPM1). J Biol. Chem. 282:14328-14336.
52. Zhu, S et al. (2008) MicroRNA-21 targets tumor suppressor genes in invasion and metastasis. Cell Res 18:350-359.
53. Frankel, L B et al. (2008) Programmed cell death 4 (PDCD4) is an important functional target of the microRNA miR-21 in breast cancer cells. J Biol. Chem. 283:1026-1033.
54. Asangani, I A et al. (2007) MicroRNA-21 (miR-21) post-transcriptionally downregulates tumor suppressor Pdcd4 and stimulates invasion, intravasation and metastasis in colorectal cancer. Oncogene
55. Zhu, S et al. (2008) MicroRNA-21 targets tumor suppressor genes in invasion and metastasis. Cell Res 18:350-359.
56. Sayed, D et al. (2008) MicroRNA-21 Targets Sprouty2 and Promotes Cellular Outgrowths. Mol. Biol. Cell
57. Yanaihara, N et al. (2006) Unique microRNA molecular profiles in lung cancer diagnosis and prognosis. Cancer Cell 9:189-198.
58. Schetter, A J et al. (2008) MicroRNA expression profiles associated with prognosis and therapeutic outcome in colon adenocarcinoma. JAMA 299:425-436.
59. Pekarsky, Y et al. (2006) Tc11 expression in chronic lymphocytic leukemia is regulated by miR-29 and miR-181. Cancer Res 66:11590-11593.
60. Taganov, K D, Boldin, M P, Chang, K J, Baltimore, D (2006) NF-kappaB-dependent induction of microRNA miR-146, an inhibitor targeted to signaling proteins of innate immune responses. Proc Natl Acad Sci U.S.A 103:12481-12486.
61. Seike, M et al. (2007) A cytokine gene signature of the lung adenocarcinoma and its tissue environment predicts prognosis. J Natl Cancer Inst 99:1257-1269.
62. Croce, C M, Calin, G A (2005) miRNAs, cancer, and stem cell division. Cell 122:6-7.
63. Calin, G A, Croce, C M (2006) MicroRNA signatures in human cancers. Nat. Rev. Cancer 6:857-866.
64. Lodish, H F, Zhou, B, Liu, G, Chen, C Z (2008) Micromanagement of the immune system by microRNAs. Nat. Rev. Immunol. 8:120-130.
65. Lindsay, M A (2008) microRNAs and the immune response. Trends Immunol.
66. Hussain, S P, Harris, C C (2007) Inflammation and cancer: an ancient link with novel potentials. Int. J Cancer 121:2373-2380.
67. Lawrie, C H et al. (2007) MicroRNA expression distinguishes between germinal center B cell-like and activated B cell-like subtypes of diffuse large B cell lymphoma. Int. J. Cancer 121:1156-1161.
68. Fulci, V et al. (2007) Quantitative technologies establish a novel microRNA profile of chronic lymphocytic leukemia. Blood 109:4944-4951.
69. Loffler, D et al. (2007) Interleukin-6 dependent survival of multiple myeloma cells involves the Stat3-mediated induction of microRNA-21 through a highly conserved enhancer. Blood 110:1330-1333.
70. Feber, A et al. (2008) MicroRNA expression profiles of esophageal cancer. J. Thorac. Cardiovasc. Surg. 135:255-260.
71. Guo, Y et al. (2008) Distinctive microRNA profiles relating to patient survival in esophageal squamous cell carcinoma. Cancer Res 68:26-33.
72. Chang, E Y et al. (2007) Accuracy of pathologic examination in detection of complete response after chemoradiation for esophageal cancer. Am. J. Surg. 193:614-617.
73. Mooney, M M (2005) Neoadjuvant and adjuvant chemotherapy for esophageal adenocarcinoma. J. Surg. Oncol. 92:230-238.
74. Trivers, K F et al. (2005) Demographic and lifestyle predictors of survival in patients with esophageal or gastric cancers. Clin. Gastroenterol. Hepatol. 3:225-230.
75. Sundelof, M, Lagergren, J, Ye, W (2008) Patient demographics and lifestyle factors influencing long-term survival of oesophageal cancer and gastric cardia cancer in a nationwide study in Sweden. Eur. J. Cancer
76. Liu, C G et al. (2004) An oligonucleotide microchip for genome-wide microRNA profiling in human and mouse tissues. Proc Natl Acad Sci U.S.A 101:9740-9744.
77. Ihaka R., G R (1996) R: A Language for Data Analysis and Graphics. Journal of Computational and Graphical Statistics 5:299-314.

1. A method of detecting one or more of esophageal adenocarcinoma, Barrett's esophagus and esophageal squamous cell carcinoma or a sample, the method comprising:
analyzing the sample for the altered expression of at least one biomarker associated with esophageal adenocarcinoma, Barrett's esophagus or esophageal squamous cell carcinoma, and
correlating the altered expression of the at least one biomarker with the presence or absence of esophageal adenocarcinoma, Barrett's esophagus or esophageal squamous cell carcinoma in the sample,
wherein the at least one biomarker is selected from the group consisting of the mirs listed in Table 2 (FIG. 6).

2. The method of claim 1, wherein the correlation distinguishes between one more or of:
1) cancerous tissue (CT) and non-cancerous tissue (NCT) in adenocarcinoma (ADC) patients;
2) cancerous tissue (CT) and non-cancerous tissue (NCT) in adenocarcinoma (ADC) patients with Barrett's esophagus (BE);
3) Barrett's esophagus (BE) and non-Barrett's esophagus (NBE) in adenocarcinoma patients (ADC);
4) cancerous tissue (CT) and non-cancerous tissue (NCT) in squamous cell carcinoma (SCC); and
5) adenocarcinoma (ADC) and squamous cell carcinoma (SCC) in cancerous tissue (CT).

3. The method of claim 2, wherein for correlation 1), the sample is analyzed for one or more of:
the increased expression of at least one biomarker that is selected from the group consisting of mir-21, mir-223, mir-146a, mir-146b, and mir-181a; and,
the decreased expression of at least one biomarker that is selected from the group consisting of mir-203 and mir-205.

4. The method of claim 2, wherein for correlation 2), the sample is analyzed for one or more of:
the increased expression of at least one biomarker that is selected from the group consisting of mir-21, mir-103, and mir-107; and,
the decreased expression of at least one biomarker that is selected from the group consisting of let-7c, mir-210, mir-203 and mir-205.

5. The method of claim 2, wherein for correlation 3), the sample is analyzed for one or more of:
the increased expression of at least one biomarker that is selected from the group consisting of mir-192, mir-215, mir-194, mir-135a, mir-92, mir-93, mir-7, mir-17, mir-20b, mir-107, mir-103 and mir-191; and,
the decreased expression of at least one biomarker that is selected from the group consisting of mir-30b, mir-193a, let-7b, let-71, let-7d, let-7a, mir-369 and let-7c.

6. The method of claim 2, wherein for correlation 4), the sample is analyzed for one or more of:
the increased expression of at least one biomarker that is selected from the group consisting of mir-21, mir-223, mir-146b, mir-224, mir-155, mir-7-2, mir-181b, mir-146a, mir-181, mir-7, mir-16, mir-122a, mir-125a, and mir-16; and,
the decreased expression of at least one biomarker that is selected from the group consisting of mir-202, mir-29c, mir-30b, mir-30c, mir-126, mir-99a, mir-220, mir-320, mir-499, mir-30c, mir-125b, mir-1, mir-145, mir-143, mir-378, mir-200b, mir-133a, mir-375 and mir-203.

7. The method of claim 2, wherein for correlation 5), the sample is analyzed for one or more of:
the increased expression of at least one biomarker that is selected from the group consisting of mir-215, mir-192 and mir-194; and,
the decreased expression of at least one biomarker that is selected from the group consisting of mir-142, mir-224 and mir-155.

8. The method of claim 1, wherein the sample is blood or tissue.

9. The method of claim 8, wherein the tissue is esophageal tissue.

10. The method of claim 9, wherein the esophageal tissue is selected from the group consisting of tumor tissue, nontumor tissue, and tissue adjacent to a tumor.

11. A method of early diagnosing a subject suspected of having esophageal adenocarcinoma, Barrett's esophagus or squamous cell carcinoma, the method comprising:
obtaining a sample from the subject,
analyzing the sample for the altered expression of at least one biomarker associated with esophageal adenocarcinoma, Barrett's esophagus or squamous cell carcinoma;
correlating the altered expression of at least one biomarker with the presence of esophageal adenocarcinoma, Barrett's esophagus or squamous cell carcinoma in the subject,
wherein the at least one biomarker is selected from the group consisting of the mirs listed in Table 2 (FIG. 6).

12. The method of claim 11, wherein the correlation distinguishes between one more or of:
1) cancerous tissue (CT) and non-cancerous tissue (NCT) in adenocarcinoma (ADC) patients;
2) cancerous tissue (CT) and non-cancerous tissue (NCT) in adenocarcinoma (ADC) patients with Barrett's esophagus (BE);
3) Barrett's esophagus (BE) and non-Barrett's esophagus (NBE) in adenocarcinoma patients (ADC);
4) cancerous tissue (CT) and non-cancerous tissue (NCT) in squamous cell carcinoma (SCC); and
5) adenocarcinoma (ADC) and squamous cell carcinoma (SCC) in cancerous tissue (CT).

13. The method of claim 12, wherein for correlation 1), the sample is analyzed for one or more of:
the increased expression of at least one biomarker that is selected from the group consisting of mir-21, mir-223, mir-146a, mir-146b, and mir-181a; and,
the decreased expression of at least one biomarker that is selected from the group consisting of mir-203 and mir-205.

14. The method of claim 12, wherein for correlation 2), the sample is analyzed for one or more of:
the increased expression of at least one biomarker that is selected from the group consisting of mir-21, mir-103, and mir-107; and,
the decreased expression of at least one biomarker that is selected from the group consisting of let-7c, mir-210, mir-203 and mir-205.

15. The method of claim 12, wherein for correlation 3), the sample is analyzed for one or more of:
the increased expression of at least one biomarker that is selected from the group consisting of mir-192, mir-215, mir-194, mir-135a, mir-92, mir-93, mir-7, mir-17, mir-20b, mir-107, mir-103 and mir-191; and,
the decreased expression of at least one biomarker that is selected from the group consisting of mir-30b, mir-193a, let-7b, let-71, let-7d, let-7a, mir-369 and let-7c.

16. The method of claim 12, wherein for correlation 4), the sample is analyzed for one or more of:
the increased expression of at least one biomarker that is selected from the group consisting of mir-21, mir-223, mir-146b, mir-224, mir-155, mir-7-2, mir-181b, mir-146a, mir-181, mir-7, mir-16, mir-122a, mir-125a, and mir-16; and,
the decreased expression of at least one biomarker that is selected from the group consisting of mir-202, mir-29c, mir-30b, mir-30c, mir-126, mir-99a, mir-220, mir-320, mir-499, mir-30c, mir-125b, mir-1, mir-145, mir-143, mir-378, mir-200b, mir-133a, mir-375 and mir-203.

17. The method of claim 12, wherein for correlation 5), the sample is analyzed for one or more of:
the increased expression of at least one biomarker that is selected from the group consisting of mir-215, mir-192 and mir-194; and,
the decreased expression of at least one biomarker that is selected from the group consisting of mir-142, mir-224 and mir-155.

18. The method of claim 12, wherein the sample is blood or tissue.

19. The method of claim 18, wherein the tissue is esophageal tissue.

20. The method of claim 19, wherein the esophageal tissue is selected from the group consisting of tumor tissue, nontumor tissue, and tissue adjacent to a tumor

21. A method of determining the likelihood of a subject to develop esophageal adenocarcinoma, Barrett's esophagus or esophageal squamous cell carcinoma, the method comprising:
analyzing the sample for the altered expression of at least one biomarker associated with esophageal adenocarcinoma, Barrett's esophagus or esophageal squamous cell carcinoma;
correlating the extent of altered expression of the biomarker with the likelihood that the subject will develop esophageal adenocarcinoma, Barrett's esophagus or esophageal squamous cell carcinoma;
wherein at least one biomarker is selected from the group consisting of the mirs listed in Table 2 (FIG. 6).

22. The method of claim 21, wherein the correlation distinguishes between one more or of:
1) cancerous tissue (CT) and non-cancerous tissue (NCT) in adenocarcinoma (ADC) patients;
2) cancerous tissue (CT) and non-cancerous tissue (NCT) in adenocarcinoma (ADC) patients with Barrett's esophagus (BE);
3) Barrett's esophagus (BE) and non-Barrett's esophagus (NBE) in adenocarcinoma patients (ADC);
4) cancerous tissue (CT) and non-cancerous tissue (NCT) in esophageal squamous cell carcinoma (SCC); and
5) adenocarcinoma (ADC) and squamous cell carcinoma (SCC) in cancerous tissue (CT).

23. The method of claim 22, wherein for correlation 1), the sample is analyzed for one or more of:
the increased expression of at least one biomarker that is selected from the group consisting of mir-21, mir-223, mir-146a, mir-146b, and mir-181a; and,
the decreased expression of at least one biomarker that is selected from the group consisting of let-7c, mir-203 and mir-205.

24. The method of claim 22, wherein for correlation 2), the sample is analyzed for one or more of:
the increased expression of at least one biomarker that is selected from the group consisting of mir-21, mir-103, mir-107; and,
the decreased expression of at least one biomarker that is selected from the group consisting of let-7c, mir-210, mir-203 and mir-205.

25. The method of claim 22, wherein for correlation 3), the sample is analyzed for one or more of:
the increased expression of at least one biomarker that is selected from the group consisting of mir-192, mir-215, mir-194, mir-135a, mir-92, mir-93, mir-7, mir-17, mir-20b, mir-107, mir-103 and mir-191; and,
the decreased expression of at least one biomarker that is selected from the group consisting of mir-30b, mir0193a, let-7b, let-71, let-7d, let-7a, mir-369 and let-7c.

26. The method of claim 22, wherein for correlation 4), the sample is analyzed for one or more of:
the increased expression of at least one biomarker that is selected from the group consisting of mir-21, mir-223, mir-146b, mir-224, mir-155, mir-7-2, mir-181b, mir-146a, mir-181, mir-7, mir-16, mir-122a, mir-125a, and mir-16; and,
the decreased expression of at least one biomarker that is selected from the group consisting of mir-202, mir-29c, mir-30b, mir-30c, mir-126, mir-99a, mir-220, mir-320, mir-499, mir-30c, mir-125b, mir-1, mir-145, mir-143, mir-378, mir-200b, mir-133a, mir-375 and mir-203.

27. The method of claim 22, wherein for correlation 5), the sample is analyzed for one or more of:
the increased expression of at least one biomarker that is selected from the group consisting of mir-215, mir-192 and mir-194; and,
the decreased expression of at least one biomarker that is selected from the group consisting of mir-142, mir-224 and mir-155.

28. The method of claim 21, wherein the sample is blood or tissue.

29. The method of claim 28, wherein the tissue is esophageal tissue.

30. The method of claim 29, wherein the esophageal tissue is selected from the group consisting of tumor tissue, nontumor tissue, and tissue adjacent to a tumor.

31. (canceled)

32. (canceled)

33. The method of claim 3, wherein the biomarkers are detected in the sample using at least one probe selected from the group consisting of miRNA probes listed in Supplemental Table 1 (FIG. 8).

34. The method of claim 4, wherein the biomarkers are detected in the sample using probes selected from the group consisting of miRNA probes listed in Supplemental Table 2 (FIG. 9).

35. The method of claim 5, wherein the biomarkers are detected in the sample using at least one probe selected from the group consisting of miRNA probes listed in Supplemental Table 3 (FIG. 10).

36. The method of claim 6, wherein the biomarkers are detected in the sample using at least one probe selected from the group consisting of miRNA probes listed in Supplemental Table 5 (FIG. 12).

37. The method of claim 7, wherein the biomarkers are detected in the sample using at least one probe selected from the group consisting of miRNA probes listed in Supplemental Table 8 (FIG. 15).

38. A method of comparing adenocarcinoma tissue samples that have undergone chemoradiation therapy and carcinoma tissue samples that have not undergone chemoradiation therapy, comprising:
comparing differential expression of at least one of biomarker selected from the group consisting of the mirs listed in Supplemental Table 4 (FIG. 11).

39. A method of comparing nodal involvement in squamous cell carcinoma tissue samples, comprising:
comparing differential expression of at least one of biomarker selected from the group consisting of the mirs listed in Supplemental Table 6 (FIG. 13).

40. A method of comparing staging in squamous cell carcinoma tissue samples, comprising:
comparing differential expression of at least one of biomarker selected from the group consisting of the mirs listed in Supplemental Table 7 (FIG. 14).

41. (canceled)

42. (canceled)

43. (canceled)

44. A method of diagnosing whether a subject has, or is at risk for developing, esophageal adenocarcinoma, Barrett's esophagus or esophageal squamous cell carcinoma, comprising:
reverse transcribing RNA from a test sample obtained from the subject to provide a set of target oligodeoxynucleotides;
hybridizing the target oligodeoxynucleotides to a microarray comprising miRNA-specific probe oligonucleotides to provide a hybridization profile for the test sample; and
comparing the test sample hybridization profile to a hybridization profile generated from a control sample,
wherein an alteration in the signal of at least one miRNA is indicative of the subject either having, or being at risk for developing, an esophageal adenocarcinoma, Barrett's esophagus or esophageal squamous cell carcinoma related disease;
wherein the mir is selected from the group consisting of the mirs listed in Table 2 (FIG. 6).

45. The method of claim 44, wherein the signal of at least one mir, relative to the signal generated from the control sample, is down-regulated.

46. The method of claim 44, wherein the signal of at least one mir, relative to the signal generated from the control sample is up-regulated.

47. (canceled)

48. (canceled)

49. A method of identifying an anti-esophageal related disease agent, comprising:
providing a test agent to an esophageal cell, and
measuring the level of at least one mir associated with decreased expression levels in the esophageal cell,
wherein an increase in the level of the mir in the esophageal cell, relative to a suitable control cell, is indicative of the test agent being an anti-cancer agent; wherein the mir is selected from the group consisting of the mirs listed in Table 2 (FIG. 6).

50. (canceled)

51. (canceled)

52. (canceled)

53. (canceled)

54. A method of assessing the effectiveness of a therapy to prevent, diagnose and/or treat an esophageal adenocarcinoma, Barrett's esophagus or esophageal squamous cell carcinoma, comprising:
subjecting an animal to a therapy whose effectiveness is being assessed, and
determining the level of effectiveness of the treatment being tested in treating or preventing esophageal adenocarcinoma, Barrett's esophagus or esophageal squamous cell carcinoma, by evaluating at least one mir listed in Table 2 (FIG. 6).

55. The method of claim 54, wherein the candidate therapeutic agent comprises one or more of: pharmaceutical compositions, nutraceutical compositions, and homeopathic compositions.

56. The method of claim 55, wherein the therapy being assessed is for use in a human subject.

57. (canceled)

58. (canceled)

59. (canceled)

60. A screening test for an esophageal adenocarcinoma, Barrett's esophagus or esophageal squamous cell carcinoma related disease comprising:
contacting one or more of the mirs listed in Table 2 (FIG. 6) with a substrate for such mir and with a test agent, and
determining whether the test agent modulates the activity of the mir.

61. A screening test of claim 60, wherein all method steps are performed in vitro.

62. (canceled)

63. (canceled)

64. (canceled)

65. The method of claim 13, wherein the biomarkers are detected in the sample using at least one probe selected from the group consisting of miRNA probes listed in Supplemental Table 1 (FIG. 8).

66. The method of claim 14, wherein the biomarkers are detected in the sample using probes selected from the group consisting of miRNA probes listed in Supplemental Table 2 (FIG. 9).

67. The method of claim 15, wherein the biomarkers are detected in the sample using at least one probe selected from the group consisting of miRNA probes listed in Supplemental Table 3 (FIG. 10).

68. The method of claim 16, wherein the biomarkers are detected in the sample using at least one probe selected from the group consisting of miRNA probes listed in Supplemental Table 5 (FIG. 12).

69. The method of claim 17, wherein the biomarkers are detected in the sample using at least one probe selected from the group consisting of miRNA probes listed in Supplemental Table 8 (FIG. 15).

70. The method of claim 23, wherein the biomarkers are detected in the sample using at least one probe selected from the group consisting of miRNA probes listed in Supplemental Table 1 (FIG. 8).

71. The method of claim 24, wherein the biomarkers are detected in the sample using probes selected from the group consisting of miRNA probes listed in Supplemental Table 2 (FIG. 9).

72. The method of claim 25, wherein the biomarkers are detected in the sample using at least one probe selected from the group consisting of miRNA probes listed in Supplemental Table 3 (FIG. 10).

73. The method of claim 26, wherein the biomarkers are detected in the sample using at least one probe selected from the group consisting of miRNA probes listed in Supplemental Table 5 (FIG. 12).

74. The method of claim 27, wherein the biomarkers are detected in the sample using at least one probe selected from the group consisting of miRNA probes listed in Supplemental Table 8 (FIG. 15).
